(19) United States
(12) Patent Application Publication
(10) Pub. No.: US 2014/0377846 A1
(43) Pub. Date: Dec. 25, 2014

(54) COMPOSITIONS, SYSTEMS AND METHODS FOR PROTECTING GENETICALLY MODIFIED ORGANISMS FROM UNAUTHORIZED USE OR RELEASE INTO THE ENVIRONMENT

(71) Applicants: Jason E. Barkeloo, Cincinnati, OH (US); Pablo J. Pomposiello, Madrid (ES); Daniel J. Hassett, Cincinnati, OH (US); Shengchang Su, Mason, OH (US)

(72) Inventors: Jason E. Barkeloo, Cincinnati, OH (US); Pablo J. Pomposiello, Madrid (ES); Daniel J. Hassett, Cincinnati, OH (US); Shengchang Su, Mason, OH (US)

(73) Assignee: Bacterial Robotics, LLC, Cincinnati, OH (US)

(21) Appl. No.: 14/218,605

(22) Filed: Mar. 18, 2014

Related U.S. Application Data
(60) Provisional application No. 61/790,735, filed on Mar. 15, 2013.

(51) Int. Cl. C12N 15/78

(52) U.S. Cl. CPC C12N 15/78
USPC 435/252.34

(57) ABSTRACT

Provided herein are compositions, methods and systems to prevent unauthorized use or release into the environment of genetically modified organisms or cells which comprises a) a genetically modified organism or cell and b) one or more keys, wherein contacting the genetically modified organisms or cell with the key results in viability or unviability of the genetically modified organism or cell.

### CROSS REFERENCE TO RELATED APPLICATIONS

This application claims the benefit of U.S. provisional application No. 61/790,735, filed Mar. 15, 2013, which is incorporated herein by reference in its entirety.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One (46,442 Byte ASCII (Text)) file named “Sequence_listing_ST25.txt,” created on Mar. 18, 2014.

### FIELD

The field relates to biotechnology and microbiology, particularly synthetic biology.

### BACKGROUND

In the information economy, where information and know-how can be easily acquired, unauthorized use of proprietary information, materials, and processes remains a growing problem. Various industries have found ways to cope with this problem by preventing unauthorized use and protection of their proprietary technology. For example, in the software field, theft and unauthorized use is largely prevented by a software digital rights management (DRM) system. In DRM, the users of a computer/software system are required to purchase and enter a pass code (username, password, or alpha-numeric code, etc.) to unlock and use the computer software, hardware, or service. In the computer/software use case analogy, the codes are not released until the software is purchased. This can be deployed as a “lite” version whereby the user has limited functionality until the pass code is purchased.

What is needed are compositions, systems and methods to prevent unauthorized use or release into the environment of genetically modified organisms or cells, such as bacteria, fungi, cell lines, protozoa, chromista (algae), plants, animals and viruses. Provided herein is a system that satisfies this need by providing “molecular keys” which can “unlock” or “lock” the genetically modified properties of each organism or cell, thereby preventing unauthorized use or access. The “molecular keys” can be provided to an authorized end user of the genetically modified organism or cell to facilitate their propagation and/or safe use.

### SUMMARY

The present invention relates to compositions, systems and methods of preventing growth and/or harvesting of genetically modified organisms by non-authorized users by requiring contacting of one or more keys with the genetically modified organisms which is required for growth and viability. The compositions, systems and methods as provided herein enable the prevention of unauthorized users from using proprietary bacteria, fungi, cell lines, protozoa, chromista (algae), plants, animals and viruses.

In one aspect, the invention provides a system comprising “molecular keys” that controls the activation (or inactivation) of vital and/or potentially toxic/harmful genes for genetically engineered organisms or cells.

In some embodiments, the system extends to all engineered biological systems, such as, for example protozoa, fungi, chromista (algae), plants, eukaryotes (e.g., important cell lines), prokaryotes, animals and viruses.

In one embodiment, the invention provides a system to prevent unauthorized use or release into the environment of genetically modified organisms or cells which comprises a) a genetically modified organism or cell and b) one or more keys, wherein contacting the genetically modified organisms or cell with the key results in viability or unviability of the genetically modified organism or cell.

In some embodiments, the genetically modified cell is a microorganism. In some embodiments, the microorganism is Psuedomonas aeruginosa.

In some embodiments, the genetically modified microorganism has a disruption in one or more of the following genes pilT (SEQ ID NO:1), bdlA (SEQ ID NO:4), lasI (SEQ ID NO:5), lasR (SEQ ID NO:6), nirS (SEQ ID NO:3), ftsZ (SEQ ID NO:7), pilA (SEQ ID NO:2), fliC (SEQ ID NO:8) and rets (SEQ ID NO:9). In some embodiments, a disruption in one or more of the following genes results in an altered electrogenic efficacy. In some embodiments, the electrogenic efficacy is enhanced or increased. In some embodiments, the genetically modified microorganism is used in a microbial fuel cell and the one or more keys of the system are as a feedstock to the microbial fuel cell.

In some embodiments, the genetically modified microbe is Pseudomonas aeruginosa comprising a disruption in one or more of pilT (SEQ ID NO:1), bdlA (SEQ ID NO:4), lasI (SEQ ID NO:5), lasR (SEQ ID NO:6), nirS (SEQ ID NO:3), ftsZ (SEQ ID NO:7), pilA (SEQ ID NO:2), fliC (SEQ ID NO:8) and retS (SEQ ID NO:9). In some embodiments, the genetically modified microbe is Pseudomonas aeruginosa comprising a disruption in pilT (SEQ ID NO:1), resulting in an increased electrogenic efficacy in a microbial fuel cell.

In some embodiments, at least 2 genes are disrupted and wherein said disrupted endogenous nucleotide sequences are selected from the group comprising pilT (SEQ ID NO:1), pilA (SEQ ID NO:2), nirS (SEQ ID NO:3), bdlA (SEQ ID NO:4), lasI (SEQ ID NO:5), lasR (SEQ ID NO:6), ftsZ (SEQ ID NO:7), fliC (SEQ ID NO:8) and retS (SEQ ID NO:9).

In some embodiments, the at least two genes are disrupted and are selected from the group consisting of pilT (SEQ ID NO:1) bdlA (SEQ ID NO:4); bdlA (SEQ ID NO:4) nirS (SEQ ID NO:3); bdlA (SEQ ID NO:4) lasI (SEQ ID NO:5); nirS (SEQ ID NO:3) pilT (SEQ ID NO:1); nirS (SEQ ID NO:3) lasI (SEQ ID NO:5); lasI (SEQ ID NO:5) pilT (SEQ ID NO:1); ftsZ (SEQ ID NO:7) pilT (SEQ ID NO:1); ftsZ (SEQ ID NO:7) bdlA (SEQ ID NO:4); ftsZ (SEQ ID NO:7) nirS (SEQ ID NO:3); ftsZ (SEQ ID NO:7) lasI (SEQ ID NO:5); pilT (SEQ ID NO:1) and retS (SEQ ID NO:9); pilA (SEQ ID NO:2) and retS (SEQ ID NO:9); nirS (SEQ ID NO:3) and retS (SEQ ID NO:9); bdlA (SEQ ID NO:4) and retS (SEQ ID NO:9); lasI (SEQ ID NO:5) and retS (SEQ ID NO:9); lasR (SEQ ID NO:6) and retS (SEQ ID NO:9); ftsZ (SEQ ID NO:7) and retS (SEQ ID NO:9); and fliC (SEQ ID NO:8) and retS (SEQ ID NO:9).

In some embodiments, at least three genes are disrupted and are selected from the group consisting of pilT (SEQ ID NO:1) bdlA (SEQ ID NO:4) ftsZ (SEQ ID NO:7); bdlA (SEQ ID NO:4) nirS (SEQ ID NO:3) ftsZ (SEQ ID NO:7); bdlA (SEQ ID NO:4) lasI (SEQ ID NO:5) ftsZ (SEQ ID NO:7); nirS (SEQ ID NO:3) pilT (SEQ ID NO:1) ftsZ (SEQ ID NO:7); nirS (SEQ ID NO:3) lasI (SEQ ID NO:5) ftsZ (SEQ ID NO:7); lasI (SEQ ID NO:5) pilT (SEQ ID NO:1) ftsZ (SEQ ID NO:7); pilT (SEQ ID NO:1) bdlA (SEQ ID NO:4) nirS (SEQ ID NO:3); pilT (SEQ ID NO:1) bdlA (SEQ ID NO:4) lasI (SEQ ID NO:5); and bdlA (SEQ ID NO:4) lasI (SEQ ID NO:5) nirS (SEQ ID NO:3). In some embodiments, retS (SEQ ID NO:9) is further disrupted.

In some embodiments, at least four genes are disrupted and are selected from the group consisting of pilT (SEQ ID NO:1) bdlA (SEQ ID NO:4) nirS (SEQ ID NO:3) ftsZ (SEQ ID NO:7); pilT (SEQ ID NO:1) bdlA (SEQ ID NO:4) lasI (SEQ ID NO:5) ftsZ (SEQ ID NO:7); bdlA (SEQ ID NO:4) lasI (SEQ ID NO:5) nirS (SEQ ID NO:3) ftsZ (SEQ ID NO:7) and pilT (SEQ ID NO:1) bdlA (SEQ ID NO:4) lasI (SEQ ID NO:5) nirS (SEQ ID NO:3). In some embodiments, retS (SEQ ID NO:9) is further disrupted.

In some embodiments, at least five genes are disrupted are selected from the group consisting of pilT (SEQ ID NO:1) bdlA (SEQ ID NO:4) lasI (SEQ ID NO:5) nirS (SEQ ID NO:3) ftsZ (SEQ ID NO:7). In some embodiments, retS (SEQ ID NO:9) is further disrupted.

In some embodiments, the genetically modified microorganism, e.g., the Pseudomonas aeruginosa having disruptions in one or more of the nine genes discussed above, is genetically modified to make it susceptible to the one or more keys of the systems and methods of the invention. In some embodiments, the cells are modified by placing one or more essential genes under control of the arabinose expression system. In some embodiments, the genetically modified microbe, e.g., Pseudomonas aeruginosa, having disruptions in one or more of the nine genes discussed above, is further modified by placing an essential gene under control of the zinc expression system. In some embodiments the essential gene is selected from the group consisting of dnaA, rpoD, rplF, rpoA, mscL, mreB, and asd and combinations thereof.

In some embodiments, the genetically modified microbe, e.g., Pseudomonas aeruginosa, having disruptions in one or more of the nine genes discussed above, is further modified by rendering it auxotrophic for one or more amino acids, such as threonine. In some embodiments, the genetically modified microbe, e.g., Pseudomonas aeruginosa, having disruptions in one or more of the nine genes discussed above, is further modified by engineering a repressor/co-repressor system coupled with one or more harmful gene products or agents selected from RNAses, DNAses, lytic phages and synthetic antibodies as provided herein.

In another aspect, the invention provides genetically modified organisms useful in the systems and methods of the present invention and methods of making thereof.

### BRIEF DESCRIPTION OF THE DRAWINGS

The skilled artisan will understand that the drawings, described below, are for illustration purposes only. The drawings are not intended to limit the scope of the present teachings in any way. The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 presents electrogenic test data from experiments utilizing a 2.5 cm diameter anode. The panels present voltages measured on four different channels corresponding to microbial fuel cells comprising different microbes. In all the panels voltage is indicated on the y axis in volts. Time progression is indicated on the x axis; the microbial fuel cells were monitored for 3.5 days. (A) presents voltages obtained from Shewenella; the voltage increases throughout the monitoring period, reaching almost 0.2 V. (B) (channel 1) presents voltages obtained from a genetically modified P. aeruginosa; the voltage fluctuates throughout the monitoring period with an early peak between 0.2 and 0.22 V. (C) (channel 2) presents voltages obtained from a second genetically modified P. aeruginosa; the voltage is high (0.3 V) early in the monitoring period and rapidly drops. (D) (channel 3) presents voltages obtained from a third genetically modified P. aeruginosa; the voltage fluctuates between 0.005 V and 0.05 V. (E) (channel 4) presents voltages obtained from a fourth genetically modified P. aeruginosa, a pilT mutant; the voltage starts near 0.5 V then decreases as the feedstock is consumed. The results from the fourth genetically modified P. aeruginosa indicate that the voltage produced exceeds the voltage produced in the microbial fuel cell containing Shewanella.

FIG. 2 depicts an example of a microbial fuel cell.

FIG. 3 depicts an example of a microbial fuel cell system.

FIG. 4 presents voltages obtained from a genetically modified P. aeruginosa, a PilT mutant; the voltage starts below 0.05 V and rises to nearly 0.45 V. These voltometric measurements are from a single 13 ml fuel cell.

FIG. 5 depicts a three example microbial fuel cell connected in series. This setup is shown coupled to a load.

FIG. 6 presents voltages obtained from genetically modified P. aeruginosa, a PilT mutant; the voltage starts between 0.3 and 0.5 V and rises to nearly 1 V. These voltometric measurements are from three 13 ml fuel cells connected in series.

FIG. 7 Schematic diagram of an arabinose-inducible gene system.

FIG. 8 (A) shows The “700” and “600” bacteriophage genes embedded within the Pseudomonas aeruginosa PAO1 genome. (B) Examples of plaque formation by Pf1 phage (left diagram) and transmission electron micrograph of P. aeruginosa Pf1 bacteriophage.

### DETAILED DESCRIPTION

Detailed descriptions of embodiments of the invention are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, the exemplary methods, devices, and materials are now described.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of “a” herein means “one or more” unless stated otherwise or where the use of “one or more” is clearly inappropriate. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

The present invention provides compositions, systems and methods which enable the prevention of unauthorized users from using proprietary genetically modified organisms or cells, such as bacteria, fungi, cell lines, protozoa, chromista (algae), plants, animals and viruses. The compositions, systems and methods can also prevent the unauthorized release of the genetically modified organisms or cells into the environment.

In some embodiments, the invention provides a system to prevent unauthorized use or release into the environment of genetically modified organisms or cells comprising a) a genetically modified organism or cell and b) one or more keys, wherein contacting the genetically modified organism or cell with the key results in viability or unviability of the genetically modified organism or cell.

In some embodiments, the key controls the activation (or inactivation) of vital and/or potentially toxic/harmful genes for the genetically modified organism or cells, resulting in viability or unviability of the genetically modified organism or cell.

In some embodiments, the genetically modified organism or cells is provided to a user. In some embodiments, one or more keys is provided to an authorized end user of the genetically modified organism or cells to facilitate their propagation and/or use. In some embodiments, the organism or cells have been genetically modified to make it susceptible or responsive to the key. The genetically modified organism or cells will be rendered ineffective, unusable, and/or unviable in the absence of the one or more keys.

The genetically modified organism or cells are not limited to a particular type of organism or cell and can extend to any engineered biological system, such as, for example, protozoa, fungi, chromista (algae), plants, eukaryotes (e.g., eukaryotic cells and cell lines) and prokaryotes. Compositions comprising the genetically modified organisms or cells are also provided herein.

In some embodiments, the genetically modified organism or cells used in the compositions, methods and systems of the invention, such as modified plants, fungi, chromista (algae), bacteria, viruses, protists, have utility in a wide variety of applications. Non-limiting examples include conducting agriculture (e.g., fertilizer, pollination), producing foods (e.g., biologically based foods), producing therapeutics and medicines, use of genetically modified cells or organisms as therapeutics, medical devices, producing fuels, chemicals, gases, cleaning or producing fresh water, sea water, contaminated water, treating or decomposing garbage/wastes, microbial fuel cells/electrogenic bioreactors, producing or for use in electronics (power-on-chip), conducting computing functions, data storage and retrieval using genetically modified DNA/RNA, sensing (e.g., environmental changes, temperature, pH, chemical composition), constructing materials (e.g., bio-based materials) and mining, mineral and metal retrieval.

In some embodiments, the genetically modified organisms or cells are microorganisms, including bacteria. In some embodiments, the genetically modified microorganisms are members of the genus Pseudomonas. In some embodiments, the genetically modified microorganism is P. aeruginosa.

In some embodiments, the microorganisms will have genetic modifications that allow it to perform various functions useful to an end user such as, for example, enhanced production of desirable products (e.g., alginate, enzymes, etc.) or power generation in a microbial fuel cell (MFC). See, e.g., U.S. Pat. No. 8,354,267 for a description of various genetic modifications for fuel cell and other applications, which disclosure is incorporated by reference herein in its entirety.

In some embodiments, the keys are required for cellular growth and propagation. In some embodiments, the presence of the one or more keys prevent activation of one or more factors which will trigger unviability of the cell. In some embodiments, the presence of the one or more keys will prevent activation of one or more of DNases, RNases and bacteriophage-mediated cell lysis which will result in unviability.

In some embodiments, the keys can be chemicals or signals (electrical, light, etc.) that are inert, nontoxic, and environmentally non-reactive. Their presence (or absence) limits the viability of the genetically enhanced or modified organism or cell.

In some embodiments, if the keys are absent from the feedstock of the genetically modified microorganisms (e.g., quorum sensing signaling autoinducers), or other chemical responses by the genetically modified microorganisms, triggers a virus (bacteriophage) with the ability to infect and destroy the genetically modified microorganisms by degrading its DNA and/or RNA. They are inserted or are already “hard-wired” into the genome during the building, engineering, or deployment of the keys with the genetically modified microorganisms.

The keys can comprise chemical, biological, mechanical, or physical mechanisms to control the activation (or inactivation) of the genetically modified microorganism. In some embodiments, the key is selected from the group consisting of chemical, light, temperature, pH and combinations thereof. In some embodiments, the key triggers unviability, such as death, hibernation or inactivation (or the inverse of homeostasis or growth).

In some embodiments, the key is a chemical inducer. In some embodiments, the key is selected from the group consisting of effective amounts of arabinose, zinc, threonine, tryptophan, salicylate and combinations thereof.

In some embodiments, the key is used to control expression of one or more essential genes. The essential gene which can be used is not limiting, and can include a single gene or multiple combinations of genes. In some embodiments, the essential gene is inserted into the cell under the control of a sequence that is responsive to the inducer followed by knocking out or inactivating the endogenous gene sequence. In some embodiments, the endogenous gene can be manipulated to render it responsive to the inducer. In some embodiments, the essential gene is selected from dnaA, essential for DNA replication (e.g., accession number b3702 (SEQ ID NO:26)); rpoD, essential for RNA transcription (e.g., accession number b3067 (SEQ ID NO:27)); rplF, essential for ribosomal protein synthesis (e.g., accession number b3305 (SEQ ID NO:28)); rpoA, sigma factor (e.g., accession number b3295 SEQ ID NO:29)), mscL, mechanosensitive channel (e.g., accession number b3291 (SEQ ID NO:30)), mreB, a prokaryotic actin homolog (e.g., accession number b3251 (SEQ ID NO:31)), and asd, aspartic semialdehyde dehydrogenase, an enzyme involved in lysine, threonine, and methionine biosynthesis (e.g., accession number PA3117 (SEQ ID NO:10)).

A. Arabinose Induction of an Essential Gene: The Sugar Arabinose Used as Key

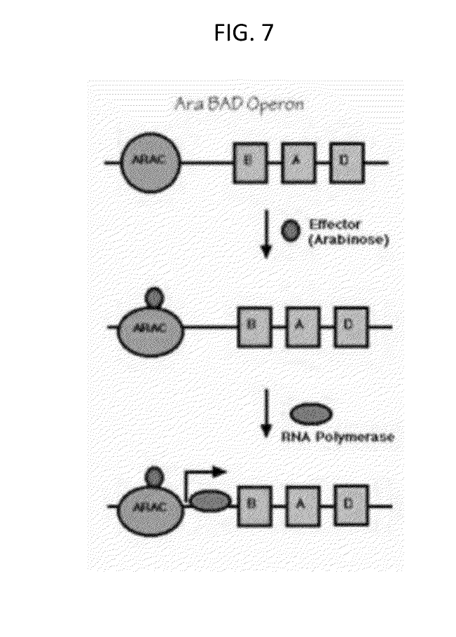

In some embodiments, the key is arabinose. In some embodiments, arabinose is used to induce expression of one or more essential genes. FIG. 7 depicts a schematic of an arabinose-inducible gene system. This system has been described in the literature (Schleif R. AraC protein, regulation of the 1-arabinose operon in Escherichia coli, and the light switch mechanism of AraC action. FEMS Microbiol Rev. 2010 September; 34(5):779-96) and used for the artificial regulation of genes of interest (Brautaset T, Lale R and Valla S. Positively regulated bacterial expression systems. Microb Biotechnol. 2009 January; 2(1):15-30). In some embodiments, arabinose can be used to trigger transcriptional activation of any gene essential that is required for viability even in rich media. In some embodiments, arabinose is used to control the expression of one or more essential genes selected from the group consisting of dnaA, rpoD, rplF, rpoA, mscL, mreB, and asd. In some embodiments, one or more of the aforementioned genes are placed under control of arabinose. In some embodiments, the asd gene (e.g., of Pseudomonas aeruginosa) is placed under the control of arabinose.

In the absence of arabinose, the AraC repressor prevents transcription of the araBAD genes. However, in the presence of arabinose, the AraC repressor becomes an activator, thus allowing for transcription of the araBAD genes in a tightly regulated fashion. If the araBAD genes are replaced by an essential gene, such as any of the ones listed above, then the expression of such gene will be dependent on the presence of arabinose, in a dose-dependent manner. Alternatively, in the absence of arabinose, the transcription will decrease and the essential gene will not be expressed. Without the essential protein, the cell will not be able to grow.

In some embodiments, a genetically modified microbe selected from the group consisting of Escherichia coli, Salmonella typhimurium and P. aeruginosa is manipulated to render it viable only in the presence of arabinose. In some embodiments, the asd gene (e.g., of Pseudomonas aeruginosa) is placed under the control of arabinose. In some embodiments, a genetically modified microorganism (such as any genetically modified microbe, e.g., Pseudomonas aeruginosa for use in fuel cell applications as described herein) is modified by placing a gene selected from the group consisting of dnaA, rpoD, rplF, rpoA, mscL, mreB, and asd under control by the arabinose system.

In some embodiments, the arabinose is added to the genetically modified microbes at a concentration range of from about 0.001 to 0.5% arabinose, from about 0.001 to 0.05% arabinose, and from about 0.005 to 0.1% arabinose.

B. Zinc Induction of an Essential Gene: The Ion Zn++ Used as Key

In some embodiments, zinc ions are used as the key to induce gene expression. In some embodiments, zinc ions are used to induce expression of one or more essential genes for viability even in rich media. This system has been described in the literature (Perron K, Caille O, Rossier C, Van Delden C, Dumas J L, Kohler T. CzcR-CzcS, a two-component system involved in heavy metal and carbapenem resistance in Pseudomonas aeruginosa. J Biol. Chem. 2004 279(10):8761-8; Liang Yang, Lin Chen, Lixin Shen and Kangmin Duan. Construction of a tightly-controlled expression system for use in Pseudomonas. Biotechnol Lett (2013) 35:1901-1906). The promoter of the operon czcCBA (accession numbers PA2520-PA2522; SEQ ID NO:11) is tightly controlled by zinc in P. aeruginosa PAO1 (Perron K, Caille O, Rossier C, Van Delden C, Dumas J L, Kohler T. CzcR-CzcS, a two-component system involved in heavy metal and carbapenem resistance in Pseudomonas aeruginosa. J Biol. Chem. 2004 279(10):8761-8). The promoter activity is completely repressed in the absence of Zinc, but is expressed in the presence of Zinc in a dose-dependent manner.

In the absence of Zinc, the CzcR repressor prevents transcription of the czcCBA genes. However, in the presence of Zn, the CzcR repressor losses the ability to repress, thus allowing for transcription of the czcCBA genes in a tightly regulated fashion. If the czcCBA genes are replaced by an essential gene, then the expression of such gene will be under the control of the czcCBA promoter, and therefore dependent on the presence of Zn. Alternatively, in the absence of Zn, the transcription will decrease and the essential gene will not be expressed. Without the essential protein, the cell will not be able to grow.

In some embodiments, a genetically modified microbe selected from the group consisting of Escherichia coli, Salmonella typhimurium and P. aeruginosa is manipulated to render it viable only in the presence of zinc. In some embodiments, the asd gene (e.g., of Pseudomonas aeruginosa) is placed under the control of zinc. In some embodiments, a genetically modified microorganism (such as any genetically modified Pseudomonas aeruginosa for use in fuel cell applications as described herein) is modified by placing a gene selected from the group consisting of dnaA, rpoD, rplF, rpoA, mscL, mreB, and asd under control by the zinc system.

The source of zinc is not limiting. In some embodiments, the source of zinc includes one or more zinc compounds and/or zinc salts. In some embodiments, the source of zinc is selected from the group consisting of zinc chloride, zinc oxide and zinc sulfate and combinations thereof. In some embodiments, the zinc is added to the genetically modified microbes at a concentration that is equivalent to the amount of zinc in a concentration of about 25 μM to about 1000 μM zinc sulfate. In some embodiments, the zinc is added to the genetically modified microbes at a concentration that is equivalent to the amount of zinc in a concentration of about 100 μM to about 400 μM zinc sulfate.

C. Amino Acid Auxotrophy: A Nutritional Requirement as Key

In some embodiments, microorganisms are rendered auxotrophs for one or more amino acids and the amino acids are used as keys. In some embodiments, the key is threonine. In some embodiments, threonine is required because one or more biosynthetic genes required for threonine synthesis are deleted in the genetically modified microorganism, such as E. coli, B. subtilis or P. aeruginosa. In some embodiments, the thrA gene is rendered non-functional (e.g., E. coli gene accession number gene b0002 (SEQ ID NO:32)), a modification that renders it a threonine auxotroph. In some embodiments, P. aeruginosa will have a deletion in the PAO1 genes thrB and thrH, accession numbers PA5495 (SEQ ID NO: 33) and PA1757 (SEQ ID NO:34), respectively. This thrB/thrH double deletion renders P. aeruginosa a threonine auxotroph. Thus, these bacteria can only grow in the presence of exogenous threonine. In this embodiment, the amino acid threonine is used as key.

In some embodiments, the amount of threonine that is added ranges from about 1 μM to about 1000 μM threonine, about 50 μM to about 600 μM threonine, or about 100 μM to about 400 μM threonine.

In some embodiments, a genetically modified microbe selected from the group consisting of Escherichia coli, B. subtilis, Salmonella typhimurium and P. aeruginosa is manipulated to render it viable only in the presence of an amino acid such as threonine. In some embodiments, a genetically modified microorganism (such as any genetically modified Pseudomonas aeruginosa for use in fuel cell applications as described herein) is modified by rendering it auxotrophic for one or more amino acids, such as threonine.

D. Pro-Phage Induction, DNases and/or RNases Under Negative Control of a Repressor/Co-Repressor Genetic Switch: A Co-Repressor Used as Key

In some embodiments, the invention provides an engineered system for genetic control of cell growth under an externally supplied key, wherein one or more deleterious proteins are placed under negative transcriptional control of a genetic repressor/co-repressor complex. In some embodiments, the co-repressor is used as a key and is a small diffusible molecule. In this embodiment, the key blocks the expression of a gene that codes for a noxious product to the cell. Therefore, in the absence of the key, the one or more noxious genes is de-repressed, the noxious protein is synthesized inside the cell, and the cell can not grow or losses viability.

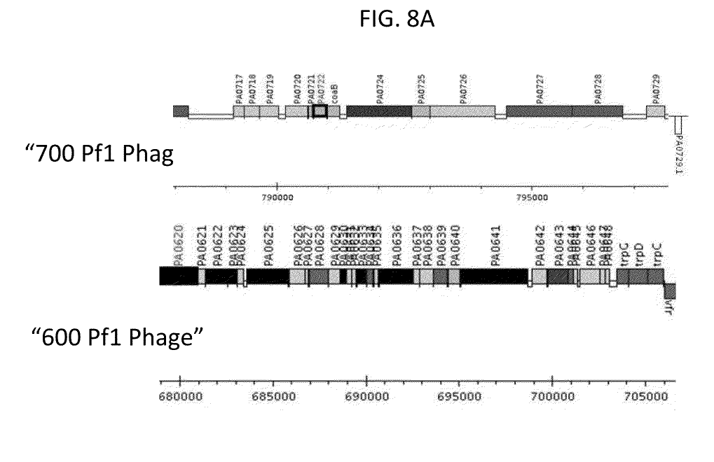

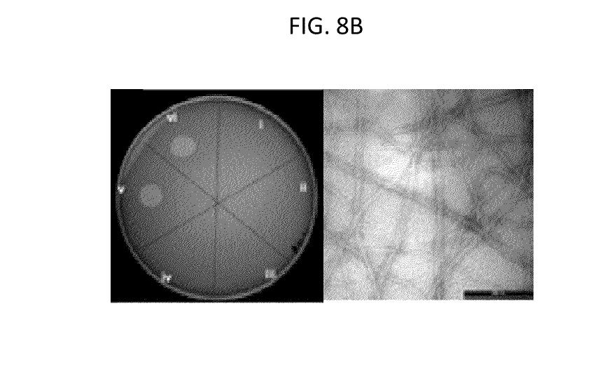

In some embodiments, the key controls the induction of prophages of P. aeruginosa into lytic phages. The P. aeruginosa genome harbors two operons that encode PF1 (P-pilus, F-flagellum) bacteriophages (see FIG. 8) (The accessory genome of Pseudomonas aeruginosa. Kung V L, Ozer E A and Hauser A R. Microbiol. Mol Biol Rev. 2010 December; 74(4):621-41). These lysogenic phages can be activated by a variety of environmental stresses. In some embodiments, the gene regulation and the induction of these prophages is artificially controlled by a repressor/co-repressor genetic switch (see below). Therefore, in the absence of the co-repressor key, the phages are induced, the lytic functions of the phage expressed and the cell lyses.

In some embodiments, the noxious gene product is a DNAse, such as DNAseI or DNAse V (e.g., accession numbers EG11481 (SEQ ID NO:35) and EG11915 (SEQ ID NO:36), respectively), placed under the negative control of a repressor/co-repressor switch (see below). In this embodiment, the absence of the co-repressor/key results in the de-repression of the DNAse-encoding gene(s), the DNAse is expressed and the host cell's DNA is degraded, which results in the inability to grow and/or loss of viability.

In some embodiments, the noxious gene product is a RNAse, such as RNAaseI or RNAseE (e.g., accession numbers EG10856 (SEQ ID NO:37) and EG10859 (SEQ ID NO:38), respectively), placed under the negative control of a repressor/co-repressor switch (see below). In this embodiment, the absence of the co-repressor key results in the de-repression of the RNAse-encoding gene(s), the RNAse is expressed and the host cell's RNA is degraded, which results in the inability to grow and/or loss of viability.

In some embodiments, the repressor/co-repressor used in the negatively-regulated genetic switch are selected from:

1. The tryptophan biosynthesis operon trpEDCBA from E. coli, which uses the amino acid tryptophan as co-repressor for the transcriptional regulator TrpR (Merino E, Jensen R A, Yanofsky C. Evolution of bacterial tip operons and their regulation. Curr Opin Microbiol. 2008 April; 11(2):78-86). In this embodiment, the key is the amino acid tryptophan. In some embodiments, the amount of tryptophan that is added ranges from about 0.001 μM to about 1000 μM tryptophan, about 0.50 μM to about 500 μM tryptophan, or about 10 μM to about 100 μM tryptophan.

2. The marRAB operon from E. coli, which uses the small molecule salicylate as a co-repressor for the transcriptional regulator MarA (Rosner J L, Storz G. Regulation of bacterial responses to oxidative stress. Curr Top Cell Regul. 1997; 35:163-77). In this embodiment, the key is the salt sodium salicylate. In some embodiments, the amount of sodium salicylate that is added ranges from about 0.001 μM to about 1000 μM sodium salicylate, about 0.50 μM to about 500 μM sodium salicylate, or about 10 μM to about 100 μM sodium salicylate.

E. Synthetic Antibodies as Growth Inhibitors

In some embodiments, recombinant, single-domain antibodies (Hamers-Casterman C, Atarhouch T, Muyldermans S, Robinson G, Hammers C, E. Bajyana Songa E, Bendahman N, and Hammers R. Naturally occurring antibodies devoid of light chains. Nature 363, 446-448 (1993) are generated against essential bacterial enzymes and proteins such as ribosomal proteins, DNA and RNA synthetases and cell division proteins such as DnaA, RpoD and RplF, and expressed intracellularly as repressors of essential functions (Löfblom J. Bacterial display in combinatorial protein engineering. Biotechnol J. 6(9): 1115-29 (2011)), or other essential genes described herein. The genes that code for these antibodies can be cloned into the bacterial chromosome under negative transcriptional regulation exerted by a repressor protein and a soluble, small co-repressor. In this system, the presence of the co-repressor allows the repressor's binding to DNA, which in turn blocks transcription of the gene coding for the antibody, and cell division proceeds. In the absence of the co-repressor, the repressor losses DNA-binding capacity, the antibody is expressed and an essential function is blocked, annulling growth. In some embodiments, a repressor/co-repressor system can be used to maintain the expression of the antibody in the off state in the presence of the key. In some embodiments, the repressor/co-repressor system is selected from:

1. The tryptophan biosynthesis operon trpEDCBA from E. coli, which uses the amino acid tryptophan as co-repressor for the transcriptional regulator TrpR (Merino E, Jensen R A, Yanofsky C. Evolution of bacterial trp operons and their regulation. Curr Opin Microbiol. 2008 April; 11(2):78-86). In this embodiment, the key is the amino acid tryptophan. In some embodiments, the amount of tryptophan that is added ranges from about 0.001 μM to about 1000 μM tryptophan, about 0.50 μM to about 500 μM tryptophan, or about 10 μM to about 100 μM tryptophan.

2. The marRAB operon from E. coli, which uses the small molecule salicylate as co-repressor for the transcriptional regulator MarA (Rosner J L, Storz G. Regulation of bacterial responses to oxidative stress. Curr Top Cell Regul. 1997; 35:163-77). In this embodiment, the key is the salt sodium salicylate. In some embodiments, the amount of sodium salicylate that is added ranges from about 0.001 μM to about 1000 μM sodium salicylate, about 0.50 μM to about 500 μM sodium salicylate, or about 10 μM to about 100 μM sodium salicylate.

### Microbial Fuel Cell Applications

In some embodiments the genetically modified cells or organisms are microbes. In some embodiments, the genetically modified microbes are useful in microbial fuel cells.

In some embodiments of a microbial fuel cell, a microbe donates electrons to an anode rather than the natural recipient molecule such as oxygen, nitrate, or sulfate. Various types of microbes including bacteria and fungi have been demonstrated to generate electrical energy during metabolism, but microbial fuel cells most commonly utilize bacteria such as Geobacter or Shewanella. Geobacter cells respond to high microbial density in such a way as to interfere with large surface area biofilm formation.

In some embodiments of microbial fuel cells, metabolic processes in the microbe generate energy in the form of electrons, especially in the anaerobic biofilm mode of growth. Rather than utilizing the energy, in a microbial fuel cell the microbe donates the electrons from a myriad of metabolized substrates to the anode for transfer through an electrical circuit. The electrical circuit carries electricity through a load, which represents work to be performed by the electron flow. The load may be a light emitting device, machinery, LCD, electrical appliance, battery charger, and many other devices.

Generally, microbes such as bacteria utilize a coenzyme known as nicotinamide adenine dinucleotide or NAD⁺ to accept electrons from, and thus oxidize, a feedstock or substrate. The NAD⁺ cleaves two hydrogen atoms from a reactant substrate. The NAD⁺ accepts one of the hydrogen atoms to become NADH and gains an electron in the process. A hydride ion, or cation, is released. The equation is as shown below, where RH₂ is oxidized, thereby reducing NAD⁺ to NADH. RH₂ could represent an organic substrate such as glucose or other organic matter such as organic waste.

RH₂+NAD⁺→NADH+H⁺+R Eq. 1

NADH is a strong reducing agent that the bacteria use to donate electrons when reducing another substrate. NADH reduces the other substrate and is concurrently reoxidized into NAD⁺. In the natural state, the other substance may be oxygen or sulphate. In a microbial fuel cell the other substance may be a mediator or an anode. A mediator transfers electrons to the anode. The electrons, prevented from moving directly from the anode to the cathode, transfer to the cathode through an external electrical circuit and through the load perform useful work.

In some embodiments, the genetically modified microbes have been modified to exhibit an altered electrogenic efficacy. These microbes can be provided to a user, along with one or more keys as described herein, to prevent the unauthorized use and/or release of the genetically modified microbes into the environment.

In some embodiments, the keys are provided to a user to be added to the microbial fuel cell as a feedstock which allows for the growth or viability of the microbes within the microbial fuel cell. In some embodiments, the keys also protect from activating DNases, RNases and/or bacteriophage-mediated cell lysis. For example, if proprietary microorganisms were stolen, the unauthorized user would not know the components within the microbial fuel cell that allow for growth and/or viability, but also protect it from DNase, RNase and bacteriophage-mediated cell lysis.

In some embodiments, microbial cells that can generate electrical current, but are not limited to bacteria and fungi. Bacterial cells that can transfer electrical current to an external component include, but are not limited to Synechocystis sp PCC 6803, Brevibacillus sp. PTH1, Pseudomonas sp., Psuedomonas aeruginosa (P. aeruginosa), Pseudomonas putida, Shewanella sp, Shewanella oneidensis MR-1, Shewanell putrefaciens IR-1, Shewanella oneidensis DSP10, Geobacter sp., Geobacter sulfurreducens, Geobacter metallireducens, Peletomaculum thermopropionicum, Methanothermobacter thermautotrophicus, Ochrobactrum anthropi, Clostridium butyricum EG3, Desulfuromonas acetoxidans, Rhodoferax ferrireducens, Aeromonas hydrophila A3, Desulfobulbus propionicus, Geopsychrobacter electrodiphilus, Geothrix fermentans, Escherichia coli, Rhodopseudomonas palustris, Ochrobactrum anthropi YZ-1, Desulfovibrio desulfuricans, Acidiphilium sp.3.2Sup5, Klebsiella pneumonia L17. Fungal cells that can generate electrical current from a metabolite include, but are not limited to Pichia anomala. See for example, Prasad et al. (2007) Biosens. Bioelectron. 22:2604-2610; Gorby et al. (2006) Proc. Natl. Acad. Sci. USA 103:11358-11363; Pham et at (2008) Appl. Microbiol. Biotechnol. 77:1119-1129; and El-Naggar et at (2008) Biophys J. 95:L10-L12; herein incorporated by reference in their entirety. Microbial cells that are capable of exocellular electron transfer are sometimes described as “exoelectrogens”, “electrochemically active microbes,” “electricigens”, “anode respiring microbes”, “electrochemically active bacteria”, and “anode respiring bacteria.”

By “electrogenic efficacy” is intended the capability to transfer electrons to or from an anode or a cathode. Such a transfer may be direct or indirect via a mediator. With regard to the electrogenic efficacy of a microbial cell, numerous components or characteristics of the cell impact electrogenic efficacy. A component or characteristic that impacts electrogenic efficacy is an electrogenic component or electrogenic characteristic. Such electrogenic-related characteristics include, but are not limited to, biofilm related characteristics such as biofilm forming abilities, biofilm density, tolerance for existence in a biofilm, cell packing characteristics, quorum sensing characteristic, cell growth rate, cell division rate, cell motility, substrate attachment, substrate adhesion, enzymatic processing of a feedstock, oxidation, phosphorylation, reduction, electron transfer, twitching motility, piliation, cell to cell adhesion, nanowire formation, nanowire structure, the ability to disperse from the biofilm and mediator related characteristics. Electrogenic efficacy can be measured using volt or current measuring devices known in the art (multimeters and computer-based measuring techniques). In some embodiments, the microbes have been genetically modified to affect one or more of the aforementioned properties, resulting in an altered electrogenic efficacy. In some embodiments, the electrogenic efficacy is enhanced or increased.

Microbes may obtain energy from a feedstock or material through a metabolic process. In a microbial fuel cell, the genetically modified microbes have access to a feedstock. In one embodiment, the feedstock is in the anodic chamber. In one embodiment, the feedstock is circulated past the anode. In one embodiment, it is recognized that the feedstock comprising the genetically modified microbes are replaced, removed, or reseeded and that the one or more keys provided to an authorized user can be added to the feedstock.

P. aeruginosa metabolizes a variety of feedstocks to produce energy. P. aeruginosa may utilize high nitrate organic materials including, but not limited to, sewage, fertilizer run-off, pulping plant effluent, and animal waste; and hydrocarbons such as diesel fuel and jet fuel; greenhouse gases, and solutions or gaseous material with a high nitrate concentration.

P. aeruginosa is capable of attaching directly and tightly to metal substrates by means of surface-exposed proteinaceous appendages known as pili (also referred to as nanowires). The attached pili allow electron transfer from the bacteria to the insoluble substrate, in a fashion similar to nanowires. See Yu et al. (2007) J. Bionanoscience 1:73-83, herein incorporated by reference in its entirety. P. aeruginosa forms biofilms in a variety of conditions including both aerobic and anaerobic conditions; anaerobic conditions result in improved biofilm formation (Yoon et al., 2002. Pseudomonas aeruginosa anaerobic respiration in biofilms: relationships to cystic fibrosis pathogenesis. Dev. Cell. 3: 593-603). During anaerobic conditions, electrons are donated to the anode surface. The protons (H+) then can react at the cathodic surface to yield hydrogen gas as a byproduct. In aerobic conditions, P. aeruginosa yields water as a byproduct at the cathode in a microbial fuel cell or during planktonic (free-swimming) growth.

An electrogenic component may include any polypeptides, peptides, or compounds involved in electrogenesis including but not limited to transporters, ion transporters, pilus components, membrane components, cytochromes, quorum sensors, redox active proteins, electron transfer components, pyocyanin, pyorubrin, pyomelanin, 1-hydroxy-phenazine or homogentisate, uncoupler proteins (UCPs), and enzymes, pilin, pilT (SEQ ID NO:1), bdlA (SEQ ID NO:4), lasI (SEQ ID NO:5), lasR (SEQ ID NO:6), nirS (SEQ ID NO:3), ftsZ (SEQ ID NO:7), pilA (SEQ ID NO:2), fliC (SEQ ID NO:8), and rets (nucleotide sequence encoding RetS protein SEQ ID NO:9 located in Accession: NC 002516.2).

In some embodiments of the invention the genetically modified microbe may exhibit an altered electrogenic efficacy. In some embodiments, a genetically modified microbial cell is a microbial cell stably transformed with an isolated nucleic acid molecule and that exhibits altered expression of a nucleotide sequence of interest. The isolated nucleic acid molecule may disrupt an endogenous nucleotide sequence of interest resulting in altered expression of the disrupted endogenous nucleotide sequence of interest or the isolated nucleic acid may comprise an expression cassette comprising a promoter operably linked to a heterologous nucleotide sequence of interest resulting in altered expression of the heterologous nucleotide sequence of interest. It is recognized that the genetically modified microbes may contain multiple genetic alterations or mutations that inactivate the genes; these may include a one, two, three, four, five, six, seven, eight, nine, ten, eleven, twelve, thirteen, fourteen, fifteen, sixteen, seventeen, eighteen, nineteen, twenty or more stably incorporated mutations. Such a stably incorporated mutation may introduce a heterogenous nucleotide sequence of interest or disrupt an endogenous nucleotide sequence.

By “stably transformed” is intended that the genome of the microbe has incorporated at least one copy of the isolated nucleic acid molecule. When a stably transformed microbe divides, both daughter cells include a copy of the isolated nucleic acid molecule. In some embodiments, genetically modified microbes include progeny of a stably transformed microbe. The invention encompasses isolated or substantially purified nucleic acid compositions. An “isolated” or substantially “purified” nucleic acid molecule, or biologically active portion thereof, is substantially free of other cellular material, or culture medium when produced by recombinant techniques or substantially free of chemical precursors or other chemicals when chemically synthesized. Isolated nucleic acid molecules may include vectors or plasmids purified from a host cell and fragments of a vector or plasmid purified from a host cell.

By altered expression is intended a change in expression level of the full nucleotide sequence of interest as compared to an untransfomed, unmodified, non-transgenic, or wild-type microbe. Such a change may be an increase or decrease in expression. An expression level may increase approximately 1%, 2%, 3%, 4%, 5%, 10%, 20%, 30%, 40%, 50%, 60%, 70%, 80%, 90%, 100%, 200%, 300%, 400%, 500% or more. An expression level may decrease approximately 1%, 2%, 3%, 4%, 5%, 10%, 20%, 30%, 40%, 50%, 60%, 70%, 80%, 90%, or 100%. It is recognized that altered expression also includes expression of a fragment of the nucleotide sequence of interest rather than the full length nucleotide sequence of interest.

A genetically modified cell for use in a microbial fuel cell can exhibit an altered cellular property such as, but not limited to, an altered electrogenic efficacy. Such an alteration may be an increase or decrease in the property of interest. It is recognized that an alteration in one cellular property may alter a second cellular property; it is further recognized that an increase in one property may decrease a second property, an increase in one property may increase a second property, a decrease in one property may decrease a second property, and a decrease in one property may increase a second property. An altered cellular property may be altered by 1%, 2%, 3%, 4%, 5%, 10%, 20%, 30%, 40%, 50%, 60%, 70%, 80%, 90%, 100%, 200%, 300%, 400%, 500% or more as compared to that cellular property in a non-modified microbial cell. Methods of analyzing cellular properties are known in the art.

An isolated nucleic acid molecule that disrupts an endogenous nucleotide sequence of interest may replace the endogenous nucleotide sequence of interest, may interrupt the endogenous nucleotide sequence, may replace a portion of the endogenous nucleotide sequence of interest, may replace a regulatory region controlling expression of the endogenous nucleotide sequence of interest, may interrupt the a regulatory region controlling expression of the endogenous nucleotide sequence, may delete an endogenous nucleotide sequence of interest, may delete a portion of an endogenous nucleotide sequence, may delete a regulatory region, or may delete a portion of a regulatory region.

In some embodiments, the genetically modified microbe has a disruption in one or more of the following genes pilT (SEQ ID NO:1), bdlA (SEQ ID NO:4), lasI (SEQ ID NO:5), lasR (SEQ ID NO:6), nirS (SEQ ID NO:3), ftsZ (SEQ ID NO:7), pilA (SEQ ID NO:2), fliC (SEQ ID NO:8) and retS (SEQ ID NO:9). In some embodiments, a disruption in one or more of the following genes results in an altered electrogenic efficacy. In some embodiments, the electrogenic efficacy is enhanced or increased.

In some embodiments, the genetically modified microbe is Pseudomonas aeruginosa comprising a disruption in one or more of pilT (SEQ ID NO:1), bdlA (SEQ ID NO:4), last (SEQ ID NO:5), lasR (SEQ ID NO:6), nirS (SEQ ID NO:3), ftsZ (SEQ ID NO:7), pilA (SEQ ID NO:2), fliC (SEQ ID NO:8) and retS (SEQ ID NO:9). In some embodiments, the genetically modified microbe is Pseudomonas aeruginosa comprising a disruption in pilT (SEQ ID NO:1), resulting in an increased electrogenic efficacy in microbial fuel cells.

In some embodiments, at least 2 endogenous nucleotide sequences of interest are disrupted and wherein said disrupted endogenous nucleotide sequences are selected from the group comprising pilT (SEQ ID NO:1), pilA (SEQ ID NO:2), nirS (SEQ ID NO:3), bdlA (SEQ ID NO:4), last (SEQ ID NO:5), lasR (SEQ ID NO:6), ftsZ (SEQ ID NO:7), fliC (SEQ ID NO:8) and retS (SEQ ID NO:9).

In some embodiments, the at least two disrupted endogenous nucleotide sequences are selected from the group consisting of pilT (SEQ ID NO:1) bdlA (SEQ ID NO:4); bdlA (SEQ ID NO:4) nirS (SEQ ID NO:3); bdlA (SEQ ID NO:4) last (SEQ ID NO:5); nirS (SEQ ID NO:3) pilT (SEQ ID NO:1); nirS (SEQ ID NO:3) last (SEQ ID NO:5); last (SEQ ID NO:5) pilT (SEQ ID NO:1); ftsZ (SEQ ID NO:7) pilT (SEQ ID NO:1); ftsZ (SEQ ID NO:7) bdlA (SEQ ID NO:4); ftsZ (SEQ ID NO:7) nirS (SEQ ID NO:3); ftsZ (SEQ ID NO:7) last (SEQ ID NO:5); pilT (SEQ ID NO:1) and retS (SEQ ID NO:9); pilA (SEQ ID NO:2) and retS (SEQ ID NO:9); nirS (SEQ ID NO:3) and retS (SEQ ID NO:9); bdlA (SEQ ID NO:4) and retS (SEQ ID NO:9); lasI (SEQ ID NO:5) and retS (SEQ ID NO:9); lasR (SEQ ID NO:6) and retS (SEQ ID NO:9); ftsZ (SEQ ID NO:7) and retS (SEQ ID NO:9); and fliC (SEQ ID NO:8) and retS (SEQ ID NO:9).

In some embodiments, at least three endogenous nucleotide sequences of interest are disrupted and said at least three disrupted endogenous nucleotide sequences are selected from the group consisting of pilT (SEQ ID NO:1) bdlA (SEQ ID NO:4) ftsZ (SEQ ID NO:7); bdlA (SEQ ID NO:4) nirS (SEQ ID NO:3) ftsZ (SEQ ID NO:7); bdlA (SEQ ID NO:4) lasI (SEQ ID NO:5) ftsZ (SEQ ID NO:7); nirS (SEQ ID NO:3) pilT (SEQ ID NO:1) ftsZ (SEQ ID NO:7); nirS (SEQ ID NO:3) lasI (SEQ ID NO:5) ftsZ (SEQ ID NO:7); lasI (SEQ ID NO:5) pilT (SEQ ID NO:1) ftsZ (SEQ ID NO:7); pilT (SEQ ID NO:1) bdlA (SEQ ID NO:4) nirS (SEQ ID NO:3); pilT (SEQ ID NO:1) bdlA (SEQ ID NO:4) lasI (SEQ ID NO:5); and bdlA (SEQ ID NO:4) lasI (SEQ ID NO:5) nirS (SEQ ID NO:3). In some embodiments, retS (SEQ ID NO:9) is further disrupted.

In some embodiments, at least four endogenous nucleotide sequences of interest are disrupted and said at least four disrupted endogenous nucleotide sequences are selected from the group consisting of pilT (SEQ ID NO:1) bdlA (SEQ ID NO:4) nirS (SEQ ID NO:3) ftsZ (SEQ ID NO:7); pilT (SEQ ID NO:1) bdlA (SEQ ID NO:4) lasI (SEQ ID NO:5) ftsZ (SEQ ID NO:7); bdlA (SEQ ID NO:4) lasI (SEQ ID NO:5) nirS (SEQ ID NO:3) ftsZ (SEQ ID NO:7) and pilT (SEQ ID NO:1) bdlA (SEQ ID NO:4) lasI (SEQ ID NO:5) nirS (SEQ ID NO:3). In some embodiments, retS (SEQ ID NO:9) is further disrupted.

In some embodiments, at least five endogenous nucleotide sequences of interest are disrupted and said at least five disrupted endogenous nucleotide sequences are selected from the group consisting of pilT (SEQ ID NO:1) bdlA (SEQ ID NO:4) lasI (SEQ ID NO:5) nirS (SEQ ID NO:3) ftsZ (SEQ ID NO:7). In some embodiments, retS (SEQ ID NO:9) is further disrupted.

In some embodiments, the genetically modified microbe, e.g., Pseudomonas aeruginosa, having disruptions in one or more of the nine genes discussed above is further modified by placing a gene selected from the group consisting of dnaA, rpoD, rplF, rpoA, mscL, mreB, and asd under control by the arabinose system. In some embodiments, the genetically modified microbe, e.g., Pseudomonas aeruginosa, having disruptions in one or more of the nine genes discussed above is further modified by placing a gene selected from the group consisting of dnaA, rpoD, rplF, rpoA, mscL, mreB, and asd under control by the zinc system. In some embodiments, the genetically modified microbe, e.g., Pseudomonas aeruginosa, having disruptions in one or more of the nine genes discussed above is further modified by rendering it auxotrophic for one or more amino acids, such as threonine. In some embodiments, the genetically modified microbe, e.g., Pseudomonas aeruginosa, having disruptions in one or more of the nine genes discussed above is further modified by engineering a repressor/co-repressor system coupled with one or more harmful gene products or agents selected from RNAses, DNAses, lytic phages and synthetic antibodies as discussed herein.

In some embodiments, the genetically modified microbes have a reduced proliferative capability as compared to a non-modified cell. In some embodiments, the genetically modified microbes have a reduced virulence as compared to a non-modified cell. In some embodiments, the genetically modified microbes have reduced motility as compared to a non-modified cell. In some embodiments, the genetically modified microbes have exhibits altered pilus sticking as compared to a non-modified cell. In some embodiments, the genetically modified microbes have altered twitching motility as compared to a non-modified cell.

In some embodiments, the genetically modified microbes exhibit an increased current output/bacterial cell when said bacterial cell is a component of a microbial fuel cell. In some embodiments, the genetically modified microbes exhibit increased electron transfer to an anode. In some embodiments, said electron transfer is direct or indirect.

The pilT gene encodes a polypeptide involved in regulating the number of pili on the bacterial surface; the protein, an electrically conductive polypeptide, is also known as the twitching motility protein. Twitching motility is the movement of bacteria by extending the pili, attaching the pili to an inanimate or animate surface and retracting the pili. Certain pilT mutants, such as pilT disruptions, exhibit reduced twitching motility and increased piliation or hyperpiliation. These pilT mutants exhibit improved attachment, cell to cell adhesion, and biofilm formation. Certain pilT mutants exhibit decreased virulence and decreased ability to detach from surfaces. While not limited by mechanism, reduced twitching motility appears to increase attachment and cell to cell attachment thus improving biofilm formation and increasing biofilm thickness. See Chaing & Burrows (2003) J. Bacterio. 2374-2387, herein incorporated by reference in its entirety.

bdlA or biofilm dispersion locus A is involved in bacterial dispersion from biofilms. As it is desirable to maintain biofilms on anodic surfaces, altering the bacterial cells ability to perform chemotaxis may improve biofilm formation and maintenance. Chemotaxis is the process of bacterial movement toward or away from a variety of stimuli or repellents. Disruption or deletion of bdlA reduces the bacterial cell's ability to detach from a surface, thus improving biofilm formation and maintenance and increasing electron transfer to the anode. See Morgan et at (2006) J. Bacteriol. 7335-7343, herein incorporated by reference in its entirety.

The fliC gene encodes a polypeptide involved in swimming motility and chemotaxis. FliC disruption mutants do not have a flagellum; thus their motility is reduced. FliC disruption mutations exhibit reduced chemotaxis and improved biofilm formation. While not being limited by theory, FliC disruption mutants may transfer more electrons to an anode.

LasI encodes N-(3-oxododecanoyl)-L-homoserine lactone synthase, a polypeptide that, while not being limited by mechanism, may be involved in the process of cell to cell signaling known as quorum sensing. Certain N-(3-oxododecanoyl)-L-homoserine lactone synthase mutants have altered biofilm characteristics. These altered biofilm formation characteristics include, but are not limited to, thinner, more compact biofilms, increased cell density, altered surface attachment properties, altered polysaccharide production, decreased polysaccharide production, and altered production of pyocyanin. Pyocyanin is redox-active, exhibits antibiotic activity, and may function as a mediator of electron transfer. Deletion of lasI also alters virulence of the bacterial cell in both animal and human cells. Such an altered virulence may be a decreased virulence in a human or animal cell. See Davies et at (1998) Science, herein incorporated by reference in its entirety.

Deletion of lasR alters virulence of the bacterial cell in both animal and human cells. Such an altered virulence may be a decreased virulence in a human or animal cell. By virulence is intended the relative capacity of a pathogen to overcome a target's defenses. Microbial cells may infect any other living organism; a particular type of microbial cell may have a limited range of targets. Pseudomonas aeruginosa is capable of infecting a wide range of targets including plants, insects, mammals. Exemplary mammals include, but are not limited to humans, bovines, simians, ovines, caprines, swines, lapines, murines and camellids. Aspects of virulence include but are not limited to the scope of suitable targets, infectivity, multiplicity of infection, transfer speed from one target to another, target cell binding ability, antibiotic sensitivity, pathogenesis and antigen production. It is recognized that lowering one aspect of virulence may not impact another aspect of virulence or may increase another aspect of virulence.

NirS encodes respiratory nitrate reductase (NIR) precursor. Inactivated nirS mutants exhibit an altered ability to survive anaerobic culture in biofilms (Yoon et at (2002) Dev Cell 3:593, herein incorporated by reference in its entirety.). While not being limited by mechanism, NIR may be the second enzymatic step in the overall process of nitrate reduction to nitrogen gas during anaerobic respiration. The product of respiratory NIR is nitric oxide (NO), a compound that is inherently toxic to bacteria in micromolar concentrations. NIR may catalyze both the one electron reduction of NO₂⁻ to NO and may catalyze the four-electron reduction of O₂ to 2H₂O. Inactivation of nirS may reduce problems associated with NO in anaerobic biofilms, increase electron flow through the pili, and reduce production of nitrous oxide (N₂O). The surface-exposed Type III secretion apparatus of a nirS mutant generates lower toxin concentrations than wild-type bacteria; nirS mutants exhibit improved virulence properties. See Van Alst, N. E. et al., 2009. Nitrite reductase NirS is required for type III secretion system expression and virulence in the human monocyte cell line THP-1 by Pseudomonas aeruginosa Infect Immun 77: 4446-4454, herein incorporated by reference in its entirety.

RetS encodes a hybrid sensor kinase/response regulator with an unconventional arrangement of functional domains. Genome-wide transcriptional profiling indicates that the retS gene is required for expression of the Type III secretion system and other virulence factors and for repression of genes responsible for exopolysaccharide components of the P. aeruginosa biofilm matrix. These disparate phenotypes are suppressed by transposon insertions in genes encoding the GacS/GacA/rsmZ signal transduction pathway, a highly conserved system involved in the control of diverse adaptive functions. RetS is a pleiotropic regulator of multiple virulence phenotypes that orchestrates genes required for acute infection and genes associated with chronic persistence. Goodman, A. (2004) Dev Cell. 7(5):745-54.

In one embodiment, the genetically modified microbe is Pseudomonas aeruginosa exhibiting enhanced biofilm formation in microbial fuel cells. By “biofilm” is intended a complex surface attached growth comprising multiple cells that are typically enmeshed or embedded within a polysaccharide/protein matrix. Biofilms occur in varying thickness; such thickness may change over time and may vary in different areas of the biofilm. Preferred thickness of a biofilm is within a range between 1 μm and 300 μm, particularly between 10 μm and 200 μm and more particularly between 30 and 100 μm. Biofilms may be comprised of multiple cell types, a single cell type, or a clonal population of cells. Multiple cell types may refer to cells of different species, cells of different strains of the same species, and cells with different genetic alterations. Several biofilm-related characteristics impact electrogenic efficacy. Biofilm-related characteristics that impact electrogenic efficacy include, but are not limited to, the number of bacteria in the biofilm, the bacterial density in the biofilm, and the number of pili attached to the anode. In an embodiment a biofilm may be attached to, growing on, adhered to, coating, touching, covering or adjacent to the surface of an anode or anode chamber. The biofilm may improve survival of cells comprising the biofilm in adverse conditions including, but not limited to, non-preferred temperatures, pH ranges, heavy metal concentration and the like. Modulating the feedstock may modulate biofilm robustness.

Various substances may be added to the feedstock provided to a biofilm. Such substances may include additional organisms compatible with the genetically modified microbe, mediator compounds, antibiotic compounds, additives for regulating or modulating an inducible promoter, and biofilm optimizers. Biofilm optimizers are compounds that modulate a metabolic property of at least one of the cells present in a biofilm, such a metabolic property may impact metabolism of available substrates or physiological cooperation between microbes within the biofilm or microbial fuel cell. Antibiotics that may be added to the feedstock are selected from the group of antibiotics to which the genetically modified microbe is resistant.

Although improved biofilm formation and maintenance is desirable, it is recognized that over-production of the bacterial cell biofilm matrix may be detrimental to a microbial fuel system. For instance, overproduction of the bacterial cells may clog the microbial fuel cell, alter the environment of the microbial fuel cell, clog a filter between the anode and cathode chambers, increase the likelihood of bacterial cell death or yield a biofilm with a non-optimal thickness. Furthermore. cell division requires energy that could be transferred to the anode. Therefore, in an embodiment exogenous regulation of cell division (or cell replication) may occur. Such regulation may involve the use of inducible promoters.

By “heterologous nucleotide sequence” is intended a sequence that is not naturally occurring with the promoter sequence. While this nucleotide sequence is heterologous to the promoter sequence, it may be homologous, or native, or heterologous, or foreign, to the host cell. Heterologous nucleotide sequences of interest include, but are not limited to, nucleotide sequences of interest encoding substances that uncouple oxidation and phosphorylation. Uncoupling, interference or disruption of the normally coupled processes, oxidation and phosphorylation alters the proton gradient from the periplasmic space to the cytoplasm. For example in bacteria treated with an exogenous uncoupler such as dinitrophenol, the rate of substrate oxidation increases and electron flow to the anode may increase. Additional uncouplers include, but are not limited to, thermogenin, UCXP-1, UCP-2, and UCP-3, that would be expressed within the microbial cell. In an embodiment, a nucleic acid molecule having a nucleotide sequence encoding an uncoupling polypeptide such as, but not limited to, thermogenin, UCXP-1, UCP-2, and UCP-3 is operably linked to an inducible promoter. The bacterial cell may then be stably transformed with an expression cassette comprising an inducible promoter operably linked to an uncoupling nucleotide sequence of interest.

Anaerobic conditions may encompass both strict anaerobic conditions with no O₂ present and mild anaerobic conditions wherein the O₂ concentration occurs within a range from 0 to 15%, 0.001% to 12.5%, 0.001% to 10%, 0.001% to 7.5%, 0.001% to 5%, 0.01% to 4%, 0.01% to 3%, 0.01% to 2%, 0.01% to 1%, or 0.01% to 0.05%. Thus, bacteria in an anaerobic environment metabolize feedstock differently than in aerobic conditions. In certain embodiments aerobic conditions are desirable. In certain embodiments anaerobic conditions are desirable. P. aeruginosa rapidly utilizes oxygen, thus may generate anaerobic conditions. Anaerobic conditions may be established by utilizing an oxygen removing system. Oxygen removing enzyme systems include, but are not limited to, a glucose-glucose oxidase catalase enzymatic O₂ removal system. Glucose oxidase converts glucose to uric acid and H₂O₂. Glucose oxidase is an oxygen dependent enzyme. The glucose oxidase and catalase reactions collectively halve the oxygen concentrations in each cycle. By “maintaining” anaerobic conditions around a biofilm is intended the establishment of anaerobic condition and sustaining said anaerobic conditions for a period of time including but not limited to, 30 seconds, 1 minute, 2 minutes, 5 minutes, 10 minutes, 15 minutes, 20 minutes, 25 minutes, 30 minutes, 45 minutes, 60 minutes, 2 hours, 3 hours, 4 hours, 5 hours, 6 hours, 7 hours, 10 hours, 15 hours, 20 hours, 24 hours, 36 hours, 48 hours, 60 hours, 72 hours, 84 hours, 96 hours, 108 hours, 5 days, 6 days, 7 days, 2 weeks, 3 weeks, 4 weeks, 1 month, 2 months, 3 months, 4 months, 5 months, 6 months, and 1 year. It is recognized that intermittent periods of aerobic conditions may occur particularly with regard to maintenance or introduction of feedstock to the microbial fuel cell, such as but not limited to, when sewage enters the fuel cell.

Methods of inoculating an anodic chamber include, but are not limited to, immersion of the anode in a culture, addition of bacteria to the anodic chamber, and addition of a matrix comprising a genetically modified microbe of the instant application.

In some embodiments, the microbial fuel cell, particularly the anodic compartment, is incubated with a 17 amino acid polypeptide from the C-terminus of the PilA peptide. The terminal 17 amino acids of the PilA protein mediates attachment to a variety of surfaces and reduces biofilm formation. In an embodiment the anodic chamber is pretreated or coated with the 17-mer, but the anode is not.

By anode is intended an electron acceptor. The anode may be of planar, cylindrical, layered spiral cylindrical, curved, angled or other geometrical shape such as but not limited to, a sheet, multiple sheets, wire mesh, porous tube, and sponge-like matrix. It is recognized that it is desirable for the anode to provide a large surface area to volume ratio. The anode may be removable from the microbial fuel cell. Optimal operation of the microbial fuel cell may involve cleaning or replacement of the anode. An anode may be constructed of any suitable material including but not limited to, metal (stainless steel), carbon, carbon nanotubes, carbon nanofibers, carbon cloth, carbon paper, platinum, graphite, graphite rods, graphite felts, graphite foams, graphite pellets, reticulated vitreous carbon (RVC) 97% porous, synthetic diamond, gold, aluminum, or other electrically conductive material. A porous metal, such as sintered steel, may provide a large surface area to volume ratio for the anode. For example, the anode may be a planar surface, multiple thin plates in close proximity with each other or a rolled planar surface or mesh. It is recognized that anode shape and anode material may be modified or optimized for different utilities of the microbial fuel cell. It is recognized that anodes may exhibit high surface area, low resistance, high conductivity, or a combination thereof and may allow high bacterial growth density. Nanomaterials are typically less than 1 micron in thickness.

An anode may be connected by a wire to the cathode. Suitable substances for the wire include, but are not limited to, copper or diamond.

The cathode of a microbial fuel cell may be an electrically conductive material including but not limited to, metal (stainless steel), carbon, carbon nanotubes, carbon nanofibers, carbon cloth, carbon paper, platinum, graphite, graphite rods, graphite felts, graphite foams, graphite pellets, reticulated vitreous carbon (RVC) 97% porous, synthetic diamond, silver, gold, aluminum, or other electrically conductive material.

A barrier such as a Nafion® membrane may separate the anodic and cationic chambers. The barrier slows, decreases, or prevents electrons from moving directly from the anode to the cathode; rather, the electrons flow through the wires of the electrical circuit. The barrier may be an ionomer membrane such as but not limited to a Nafion® perfluorosulfonic acid (PFSA) membrane (DuPont Fuel Cells, Inc). Excessive deposits of the biofilm on the barrier may impair function of the microbial fuel cell. Therefore it is advisable to maintain biofilm deposits on the barrier at a moderate level. Methods of regulating biofilm deposits include, but are not limited to, regulating bacterial cell division rates and precoating the barrier with a biofilm formation inhibitor. Biofilm formation inhibitors are known in the art and include the polypeptide having the amino acid sequence of the terminal 17 amino acids of the PilA protein, also known as the PilA 17 mer. Alternatively the anode and cathode may be separable components as for instance an anodic tube that may be removable from the cathode portion of the microbial fuel cell.

In some embodiments, several microbial fuel cells could be electrically associated in series or parallel to create a battery of fuel cells. One or more of the microbial fuel cells could be disassembled and cleaned. It is recognized that one or more components of the microbial fuel cell may be cleaned. Such cleaning may involve chemical cleaning, mechanical cleaning, scavenging the biofilm utilizing species of Bdellovibrio, scavenging the biofilm utilizing a carnivorous organism such as but not limited to a fungi, or a combination thereof. Bdellovibrio, a bactivorous bacterium, feeds upon P. aeruginosa and temporarily reverses the polarity of the electrode to release bound pili.

A user of a microbial fuel cell may fabricate or obtain a microbial fuel cell. The user of the microbial fuel cell could then use electrodes proceeding from the anode and cathode to attach the fuel cell to a load. Thus, the user completes an electrical circuit from the anode through the load to the cathode. The user could, for example, by engaging a switch, cause electrical current created by the genetically modified microbes to flow through the load. The genetically modified microbes transfer electrons from the feedstock to the anode, the electrons proceed to flow through electrodes and the load to the cathode. A barrier blocks the electrons from flow through the interior of the fuel cell. Microbial fuel cells may be used in consumer electronics perhaps through a lithium/ion battery recharger or as a replacement for lithium/ion batteries. Microbial fuel cells may be used in electric plug-in automobiles or to recharge electric plug-in automobiles. Microbial fuel cells may generate power for residential and commercial buildings by tapping into organic wastes flushed from the buildings in the outgoing sewage pipes. Microbial fuel cells may be used for large waste treatment, farms, and utilities.

The microbial fuel cell electrical system may further include an ultracapacitor connected electrically in parallel in a paired system. Ultracapacitors have the advantageous ability to store power quickly and deliver it in relatively short bursts upon demand. Pairing an ultracapacitor with a fuel cell according to an embodiment of the invention enables a user of the paired system to have a continuous flow of power when beginning to start the system.

Furthermore, advantageous use can be made of the chemical reactions of the microbial fuel cell. For example, the products of the chemical reaction at the cathode may include free hydrogen gas when the microbial fuel cell is operated anaerobically. The hydrogen gas may be collected and utilized to power a classical hydrogen fuel cell. Also, microbial fuel cells emit very little carbon dioxide and may utilize carbon dioxide as a feedstock or remove it to biocarbonate by carbonic anhydrase as an attachment to the anode. The microbial fuel cell may be designed to emit usable sugars that can become an energy source for other devices. The microbial fuel cell may be operated aerobically to emit water as a byproduct. The water may be transferred to a water storage device or transferred to the external environment.

The present invention is further illustrated by the following Examples. These Examples are provided to aid in the understanding of the invention and are not to be construed as a limitation thereof.

### EXAMPLES

### Example 1

### Construction of a Genetically Modified Pseudomonas aeruginosa PAO1 and Use of Zn++ as Key for Induction of an Essential Gene

Bacterial Growth Conditions.

All cloning is conducted in Escherichia coli DH5a. Strain S17-1 is used as a donor strain for biparental mating. E. coli is grown at 37° C. in Luria-Bertani broth (LB, Life Technologies) or on LBagar plates. Pseudomonas aeruginosa PAO1 and its derivatives are cultivated aerobically at 37° C. in LB broth, on LB agar plates, or on Pseudomonas isolation agar (PIA, Difco) medium. Antibiotics are added at the following concentrations when required. For E. coli strains, ampicillin, 100 μg/ml; gentamicin, 10 μg/ml; for P. aeruginosa, carbenicillin, 200 μg/ml; tetracycline, 100 μg/ml; gentamicin, 150 μg/ml;

DNA Manipulations.

Genomic DNA isolation, PCR, restriction enzyme digestion, ligation, cloning and DNA electrophoresis are performed according to standard techniques (Maniatis T, Fritsch E F, Sambrook J (1982) Molecular cloning: a laboratory manual. Cold Spring Harbor Laboratory, Cold Spring Harbor, N.Y. and Green, M. and Sambrook, J. (2012) Molecular Cloning: A Laboratory Manual (Fourth Edition), Cold Spring Harbor Laboratory Press, Cold Spring Harbor, N.Y.). All oligonucleotide primers are synthesized by Integrated DNA Technologies (IDT). Polymerase Chain Reaction (PCR) is performed using either Choice Taq Mastermix (Denville Scientific, Inc.) or Pfu DNA polymerase (Stratagene). Plasmids are prepared using a QIAprep Spin miniprep kits (QIAGEN) as recommended by the manufacturer. DNA fragments are purified using either a QIAquick PCR purification kit (QIAGEN) or a QIAquick gel extraction kit (QIAGEN). All cloned inserts are confirmed by automated DNA sequencing performed at the DNA Core Facility of the Cincinnati Children's Hospital Medical Center. Plasmids are introduced into E. coli by CaCl₂-mediated transformation and into P. aeruginosa via biparental conjugation.

Construction and Chromosomal Integration of Promoter PczcCBA-Driven asd.

A 322 by Zn-inducible promoter of the czcCBA operon (PA2520-PA2522) (SEQ ID NO:11) of Pseudomonas aeruginosa PAO1 is amplified by PCR with forward primer PczcC5′ containing BamHI site (5′-cgggatccgcggcgcaagcaccg-3′) (SEQ ID NO:12) and reverse primer PczcC3′ containing NdeI site (5′-cgtataccatatgccgctcctcgtctgc-3′) (SEQ ID NO:13). A 1,113 bp asd gene (PA3117) (SEQ ID NO:10), encoding aspartate semialdehyde dehydrogenase, is amplified by PCR using forward primer asd5′ with an incorporated NdeI site (5′-cgtataccatatgaagcgtgtaggtctg-3′) (SEQ ID NO:14) and reverse primer asd3′ with an incorporated EcoRI site (5′-cggaattctcagcgctccagcaggat-3′) (SEQ ID NO:15). The amplified czcCBA promoter fragment is digested with BamH1 and NdeI, and ligated with the NdeI and EcoRI digested asd gene. The ligation product PczcCBA-asd is then cloned between BamHI and EcoRI sites of mini-CTX1 (Hoang T. et al. (2000) Plasmid 43(1):59-72). The resulting construct CTX1-PczcCBA-asd is transformed into E. coli strain S17-1 (Simon, R. et al. (1983) Bio-Technol 1(9):784-791). The donor strain S17-1 carrying CTX1-PczcCBA-asd is mated with P. aeruginosa PAO1 using a biparental mating protocol (Wyckoff, T. et al. (2001) Methods Enzymol 336:144-51. Transconjugants are selected on PIA plates containing tetracycline (100 μg/ml). The isolated tetracycline resistant conjugants are subjected to colony PCR using primers attB2 (5′-gtcgccgccggcgatgc-3′) (SEQ ID NO:16) and CTX1 (5′-cctcgttcccagtttgttcc-3′) (SEQ ID NO:17). The amplification of an approximately 950 bp fragment confirms the integration of the plasmid CTX1-PczcCBA-asd into the chromosome at attB site. Next, the unwanted mini-CTX1 plasmid DNA sequence is removed from the integrants by the action of Flp recombinase, contained on plasmid pFLP2 (Hoang, T. et al. (1998) Gene 212(1):77-86). Briefly, donor strain S17-1 harboring pFLP2 is mated with the P. aeruginosa integrants overnight. Conjugants are selected on PIA plates containing carbenicillin (200 μg/ml). Carbenicillin resistant conjugants are streaked out on PIA plates containing tetracycline (100 μg/ml). Tetracycline-sensitive and carbenicillin resistant candidates are then streaked out on LB agar plates containing 7% (w/v) sucrose to cure pFLP2 plasmid. Both tetracycline and carbenicillin sensitive candidates are subjected to colony PCR using a pair of primers attB4 (5′-cgccctatagtgagtcg-3′) (SEQ ID NO:18) and attB5 (5′-cgccccaacctcgctgg-3′) (SEQ ID NO:19), which yielded an approximately 450 bp fragment, confirming the removal of unwanted mini-CTX1 plasmid backbone sequence. The final candidate is designated as PAO1::attB::PczcCBA-asd, in which the expression of a wild-type copy of asd gene at chromosomal attB site is under the control of a Zn-inducible promoter PczcCBA.

Generation of P. aeruginosa Δasd mutant. A 969 bp upstream sequence (SEQ ID NO:20) and a 848 bp downstream sequence (SEQ ID NO:21) of asd gene (PA3117) are amplified by PCR using primer pair Uasd/Bm5′(5′-cgggatccaggccgatgcggtgctgc-3′) (SEQ ID NO:22) and Uasd/Pst3′ (5′-aactgcagcgcaactacacctatatg-3′) (SEQ ID NO:23), and Dasd/Pst5′ (5′-aactgcagtcgatccggcgccgtccg-3′) (SEQ ID NO:24) and Dasd/Eco3′(5′-atagaattcggtcacgctgaggctatc-3′) (SEQ ID NO:25), respectively. The amplified upstream fragment is digested with BamHI and PstI, and ligated with the amplified downstream fragment restricted with PstI and EcoRI. The ligation product is first cloned between BamHI and EcoRI sites of cloning vector pBluescript SK⁺, creating pSKΔasd. A 1,817 bp fragment containing flanking sequences of asd is then released from pSKΔasd by digestion with BamHI and EcoRI, and cloned into gene replacement vector pEX100T-KS (Schwiezer H. et al. (1995) Gene 158(1):15-22) with modified multiple cloning sites, yielding pEXΔasd. An 900-bp PstI-flanked gentamicin resistance (Gm^{R}) cassette from pUCGM (Schweizer H. (1993) Biotechniques 15(5):831-4) is inserted into the unique PstI site of pEXΔasd. The resultant construct pEXΔasd::Gm^{R} is introduced into PAO1::attB::PczcCBA-asd via S17-1-mediated biparental mating. Single crossover recombinants are selected on PIA plates containing gentamicin (150 μg/ml). Double crossover recombination event is achieved by culturing single cross-over recombinants on LB agar containing 7% (w/v) sucrose, gentamicin (150 μg/ml), and zinc sulfate (400 μM). The Δasd mutant is confirmed by PCR analysis and DNA sequencing, and designated as PAO1Δasd::attB::PczcCBA-asd.

Validation of Conditional Lethality by Zn-Regulated Expression in P. aeruginosa.

The essential gene asd encodes aspartate semialdehyde dehydrogenase genase (Asd; EC 1.2.1.11), a key enzyme in the biosynthetic pathways for diaminopimelic acid (DAP), lysine, methionine and threonine (Hoang, T. (1997) Microbiology 143(Pt 3):899-907). DAP is an essential component of the peptidoglycan of all Gram negative and some Gram-positive bacteria (Schleifer & Kandler, 1972). Unable to synthesize DAP, uncomplemented asd mutants undergo lysis. To validate the conditional lethality by Zn-regulated expression of an asd gene in P. aeruginosa and effectiveness of zinc sulfate as a key, the following experiment is performed independently for three times.
(i) Strain PAO1Δasd::attB::PczcCBA-asd is grown overnight in LB broth supplemented with 300 μM of ZnSO₄;
(ii) Overnight culture is 1:100 diluted into fresh LB broth supplemented with 300 μM of ZnSO₄. Cell growth is monitored over time;
(iii) When culture density reaches OD₆₀₀=0.4, cells are harvested by centrifugation, washed three times with fresh LB broth, and resuspended into same volume of fresh LB broth in the absence and presence of 300 μM of ZnSO₄. Cells are sampled for colony forming unit (CFU) count at zero time point. A
(iv) Incubation of cell culture is continued. Cell growth/death is monitor hourly by OD₆₀₀ as well as colony-forming units (cfu) coun.

Once the inducer ZnSO₄ is washed away, the expression of asd gene driven by the promoter PczcCBA is turned off. Cell growth will be stopped and the culture should gradually lyse; In contrast, in the presence of ZnSO₄, cells grow normally and reach saturation.

### Example 2

### Development of Static Biofilms on Simple Glass Surfaces in Feedstock

Circular glass coverslips were attached to the bottom of 35×10 mm polystyrene tissue culture dishes with small holes in the base (Falcon). The plates were exposed to UV irradiation overnight. (UV irradiation sterilizes the culture plates).

Bacterial cells were grown in Luria Bertani media (LB) overnight.

Aerobic LB, aerobic LBN (LB+1% KNO₃), or anaerobic LBN (3 ml) was placed in each tissue culture plate. The media was inoculated with 10⁷ cfu of bacterial cells. The plates were incubated at 37° C. for 24 hours. The media was removed and the plates were washed with saline buffer. LIVE/DEAD BacLight (Molecular Probes, Inc) bacterial viability stain (0.5 ml) was added to each plate. Images were acquired on a Zeiss LSM 510 laser scanning confocal unit attached to an Axiovert microscope with a 63×14 NA oil immersion objective. For two color images, samples were scanned sequentially at 488 nm and 546 nm. Syto 9 (green fluorescence) was detected through a 505-530 nm bandpass filter and propidium iodine (red fluorescence) was detected through a 560 nm longpass filter and presented in two channels of a 512×512 pixel, 8-bit image.

### Example 3

### Development of Biofilms in Circulated Feedstock

Bacteria are grown aerobically in LB at 37° C. until the stationary growth phase. Bacteria are diluted 1:50 into 1% trypticase soy broth. Flow cells are inoculated with 0.2 ml diluted bacteria. Flow cells and bacteria are incubated for 1 hour. After an hour, flow is initiated at a rate of 0.17 ml/min. The cells are incubated 3 days at room temperature. The cells are stained with a live/dead viability stain composed of SYTO 9 and propidium iodine (Molecular Probes, Inc.). Biofilm images are obtained using an LSM 510 confocal microscope (Carl Zeiss, Inc.). The excitation and emission wavelengths for green fluorescence are 488 nm and 500 nm, while those for red fluorescence are at 490 nm and 635 nm, respectively. All biofilm experiments are repeated at least 3 times. The live/dead ratios of the biofilms are calculated using the 3D for LSM (V.1.4.2) software (Carl Zeiss). Overall biofilm structure such as thickness, water channel, bacterial density (substrate coverage), roughness coefficient and total biomass in m³/m² are assessed using COMSTAT software. COMSTAT analyzes stacks of images acquired with scanning confocal laser microscopy (SCLM) to quantify the 3-dimensional nature of biofilm structures. See Heydorn et at (2000) Microbiology 146 (Pt 10):2395, herein incorporated by reference in its entirety.

### Example 4

### Construction of P. aeruginosa Deletion Mutants

The P. aeruginosa strain PAO1 is used as the starting strain for construction of deletion mutations. Classical allelic replacement techniques are used to generate mutant strains. See Hoang et at (1998) Gene 212(1):77-86) An insertional mutagenesis cassette comprising a gentamicin resistant (Gm^{R}) nucleotide sequence, a green fluorescent protein (GFP) nucleotide sequence, and FLP recombinase target (FRT) sites flanking the gentamicin resistance sequence and the GFP sequence is developed for each gene of interest. After conjugal transfer or electroporation plasmid integrants are selected. The cells are grown in media containing 6% sucrose. The sucrose promotes deletion of the target sequence of interest. Mutants are confirmed via PCR or Southern blotting. Mutant cells undergo conjugal transfer with a cell containing a FLP-recombinase expressing plasmid such as pFLP2. pFLP2 contains the sacB sequence; growth on sucrose containing media cures the bacterial cells of the sacB containing plasmid. Expression of FLP recombinase allows excision of the FRT cassette. After curing of plasmid the P. aeruginosa deletion mutant strain is gentamicin sensitive. Multiple mutations such as double and triple mutants are constructed by similar methods.

### Example 5

### High-throughput Microbial Fuel Cell Prototype

A small high-throughput microbial fuel cell (Pilus Cell) prototype was developed. A Millipore filtration apparatus of the type commonly used to collect cells on a 1 inch nitrocellulose filter was utilized to construct the Pilus Cell prototype. When used to collect cells on a filter for radioactivity measurements in a scintillation counter, a filter is placed on the sintered plastic surface of each well. The top portion of the apparatus is tightly screwed to the base portion. The top “cup” portion of the apparatus has rubber seals to prevent leakage from each well. The base portion includes a vacuum port. The Millipore filtration apparatus has 12 wells.

The filtration apparatus has been modified into a high-throughput device for screening and monitoring power generation by up to 12 different genetically engineered bacteria. Copper wires have been soldered to the base of twelve 2.54 cm×0.2 mm circular wafers of stainless steel. The milled steel was treated with acetone and then methanol to remove residual oils. The steel wafers were brushed with a wire brush to increase the surface area of the steel available for bacterial binding. The copper wire attached to the wafer represents the anode. The copper wires from each wafer were drawn through what was formerly the vacuum port of the apparatus. The copper wires were connected to a voltage/current measuring device. Each well may hold up to 15 mls of media; in these experiments 7 mls of media were used. Two holes were drilled into each of twelve grade 6 rubber stoppers that fit snugly in the wells. An 8 inch copper wire that extends 0.25 inches into the media in the anode was placed in the main hole of the stopper. This copper wire represents the cathode. This high-throughput device allows evaluation of up to 12 samples at a time. Once assembled, each well has the capacity to be an independent microbial fuel cell.

### Example 6

### High Through-Put Microbial Fuel Cell Voltage/Current Evaluation

The above described high-throughput microbial fuel cell prototype was used to evaluate voltage and current generation from wildtype Pseudomonas aeruginosa (POA), Shewanella oneidensis, and a mutant strain (pilT, bdlA, nirS, lasI, or fliC pilA). The entire high-throughput microbial fuel cell prototype was assembled and secured by a bolt on the top of the apparatus. Each well utilized in the experiment was sterilized by treatment with ethanol. The ethanol was removed and the apparatus was dried in a germ-free laminar flow hood. LB+1% KNO₃ media (7 ml) was placed in each well utilized in the experiment. A stationary phase grown aerobic culture (70 μA, a 1:100 dilution) for each bacterial sample (wildtype Pseudomonas, Shewanella, and a mutant strain) was added to the media in the well. A medium alone control well was also prepared and monitored. Rubber stoppers and copper cathode wires were treated with ethanol prior to securing the stoppers in the wells. The device was incubated at 37° C. for 24 hours under anaerobic conditions.

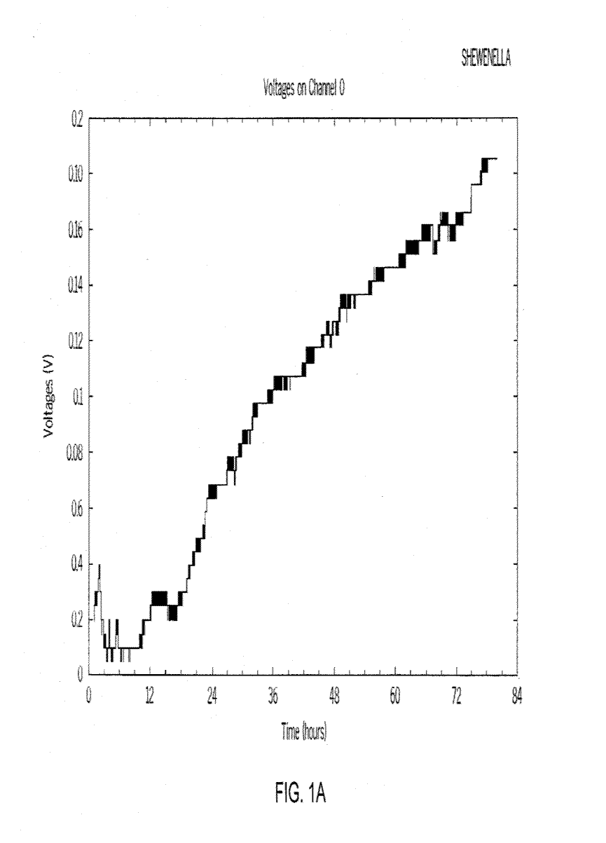

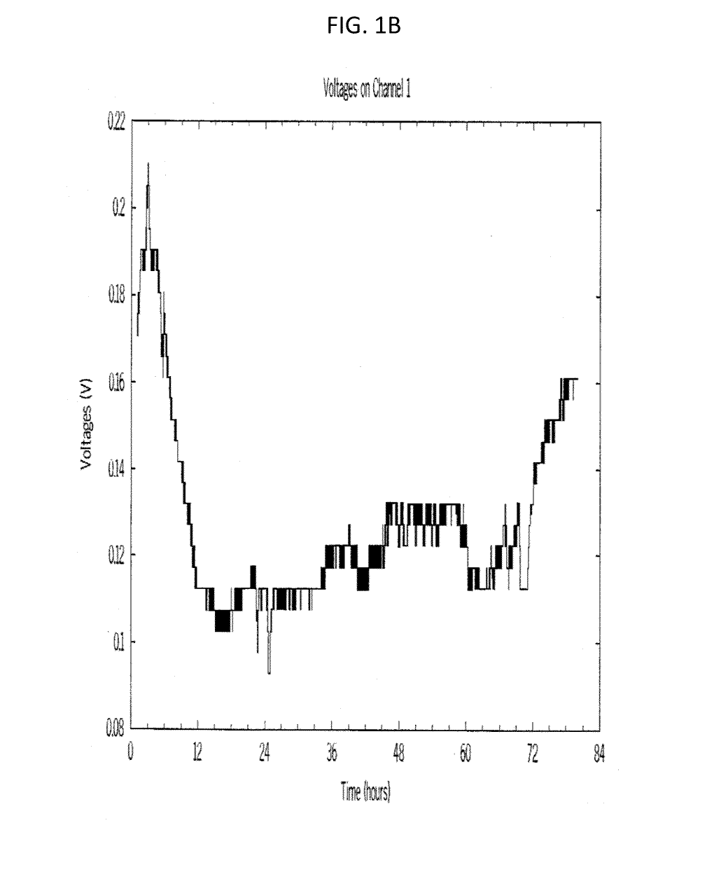

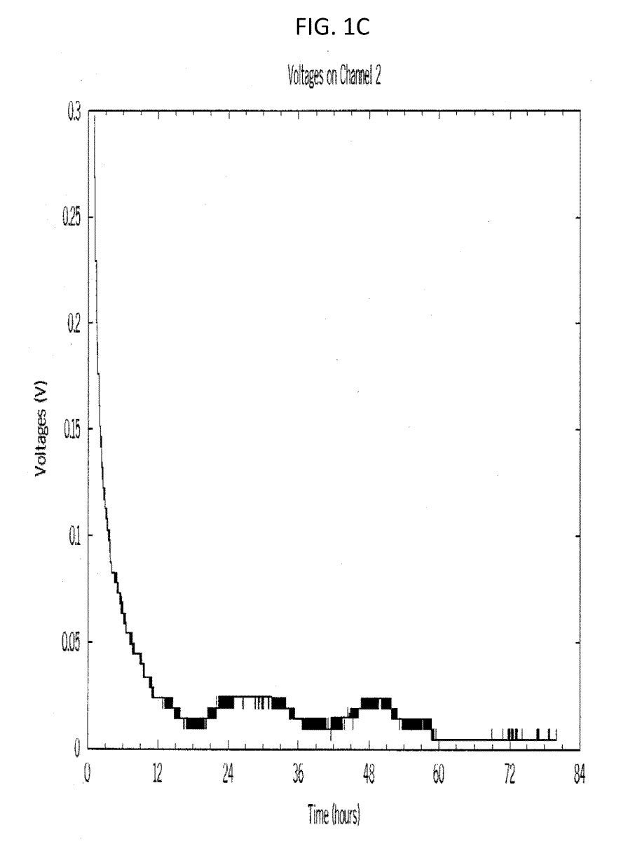

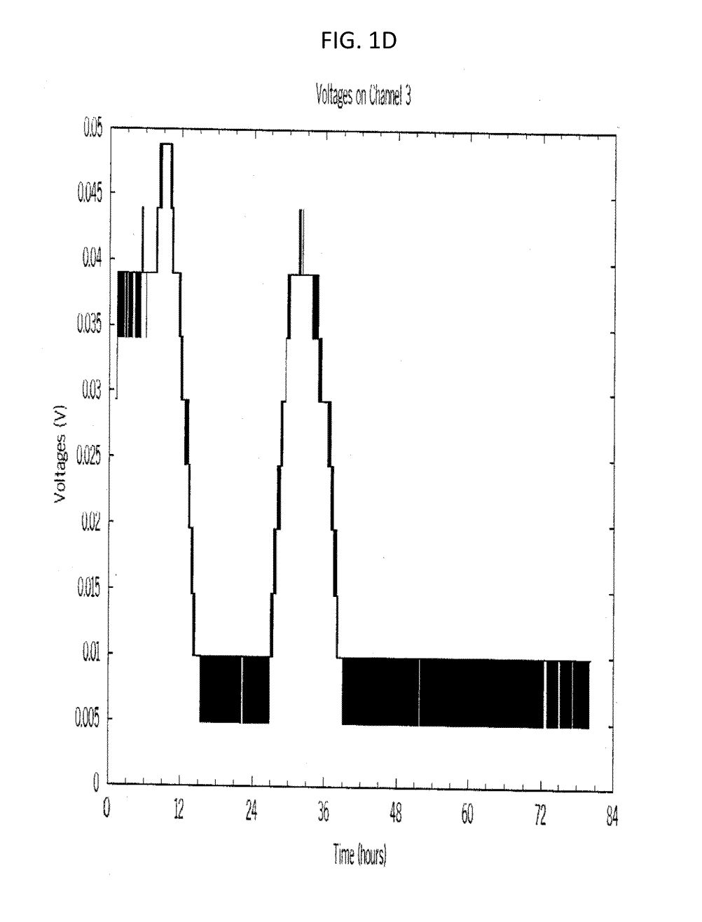

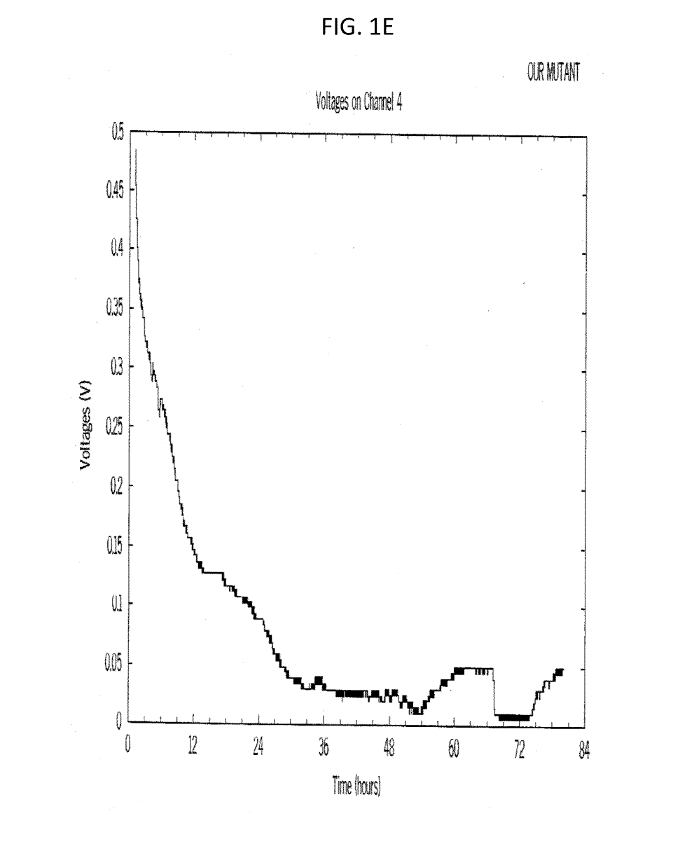

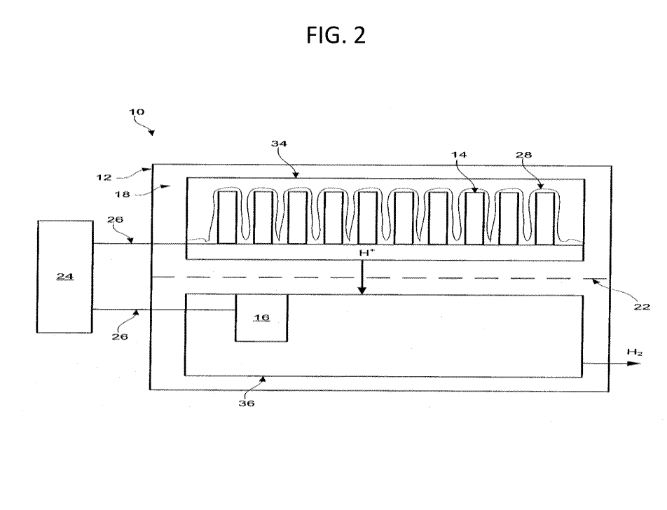

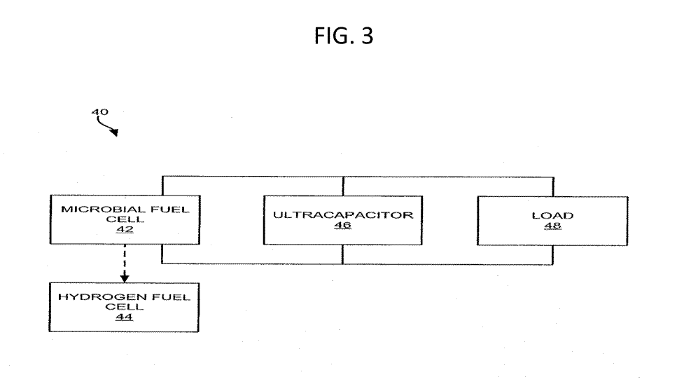

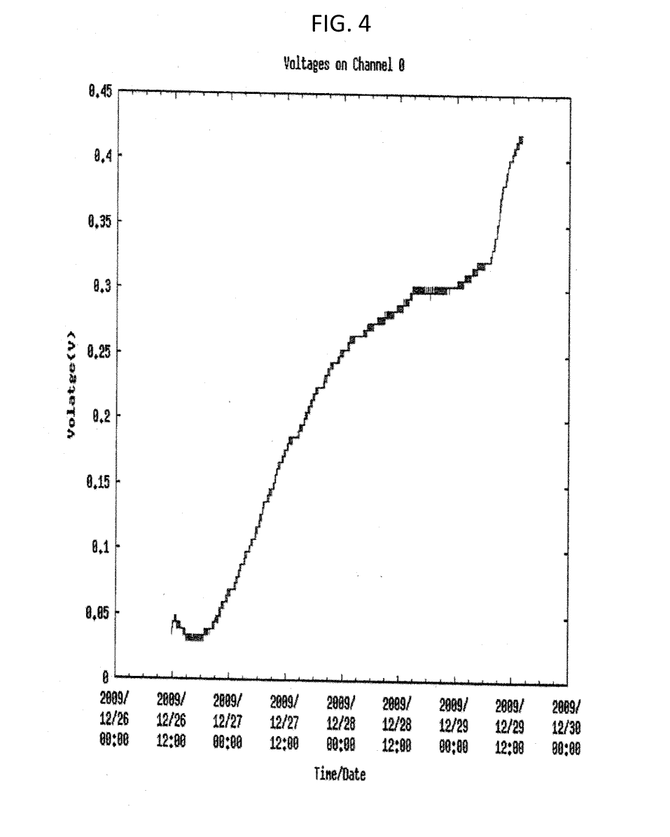

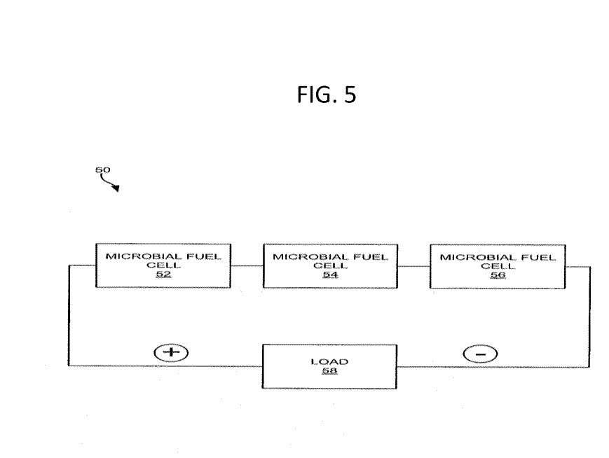

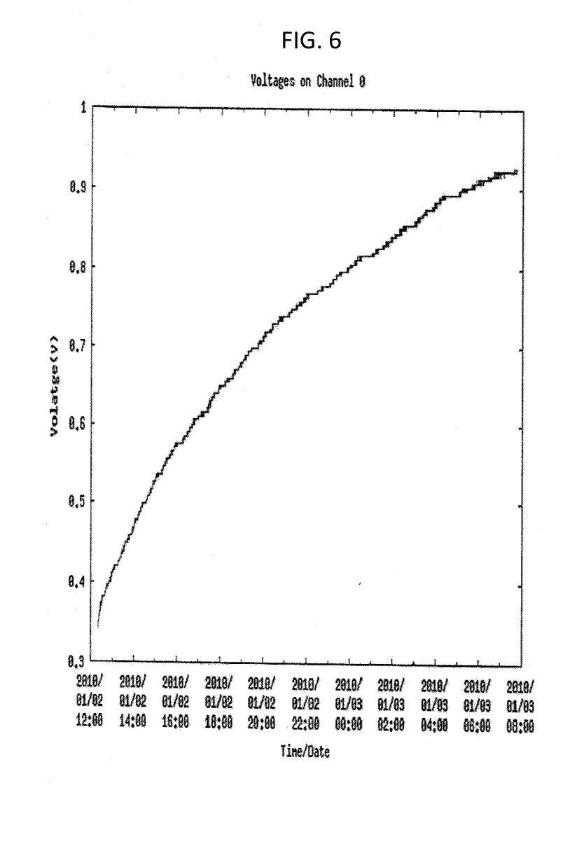

Measurements were recorded as described elsewhere herein. The stoppers were removed and the media was aspirated away. Saline (0.9%) was gently applied to each well. The saline solution was removed by aspiration. The saline wash was performed three times. Ethanol was swabbed over the plastic regions of each well. LB+1% KNO₃ media (7 ml) was added to each well. The recording process was repeated. Results from one such experiment are presented in FIG. 1.

### Example 7

### Voltage and Current Monitoring of the High-Throughput Microbial Fuel Cell Prototype

Measurements were obtained using a LabJack U12, 8 channel 12 bit USB A/D for data acquisition system. Four channels were used to monitor microbial fuel cell voltages. The 3 cm copper anodes were connected to four LT1012 high input impendence buffer amplifiers. The outputs of these amplifiers were then connected to the channel AIO-AI3 inputs of the Labjack A/D. Current measurements were made by connecting a LT1101 instrumentation amplifier across the 1K current sense resistor. By measuring the voltage drop across the resistor and utilizing Ohm's law (I=E/R) the current flowing in the cell circuit can be calculated.

The measurement system utilized allows voltage and current measurements to be done remotely via the internet. The system utilizes eight different graphic monitoring systems that can be configured to monitor various combinations of voltages and currents as dictated by the experimental design.

### Example 8

### Development of Microbial Fuel Cell with Increased Anode Surface Area

A 23-plate stainless steel 314 anode system is constructed. The first 21 plates are of the following dimensions: 0.05×9.851×7.554 inches. This involves a total surface area of 197.56 inches. The other two plates are 0.05×9.851×7.884 inches. The two larger plates serve as “legs” facing either in or toward the Nafion membrane and adding an additional 96.2 inches of surface area. Thus the total estimated surface area is approximately 294 inches. The two larger plates provide support to the 21 plate component. The electrode from the anode to the cathode compartment is stainless steel and fitted with Swagelok fittings into similar fittings embedded within the cathode.

A single plate of hot, isostatic pressed graphite (GraphiteStore.com) of 0.125×9.6×4.65 inches is used for the cathode.

After the anode is assembled, the anode is treated with 1% bleach, then 95% ethanol, and then 70% ethanol. Small 1×1×0.05 inch stainless steel wafers are used to monitor biofilm formation. The anode is incubated in a Coy anaerobic chamber in 1 liter of LB+1% KNO₃ at 37° C. for 24 hours inoculated with bacteria.

Experiments are performed with wild-type bacteria or with various mutants. The overall efficiency of the wild-type and mutant strains is compared. The complete anode assembly with a mature biofilm attached is submerged in anaerobic 0.9% NaCl solution and removed from the solution. Submersion and removal may be repeated. (Unattached bacteria are removed by this process.) The anode with the attached mature biofilm is placed in the large microbial fuel cell assembly. Two plastic boxes, one containing the anode, the other the cathode are filled with LB+1% KNO₃. The anodic and cathodic chambers are treated with glucose oxidase. Glucose oxidase converts glucose to uric acid and H₂O₂. H₂O₂ is treated with catalase. The glucose oxidase and catalase reactions lower the oxygen concentration. The anode is poised at approximately 250-400 mV (versus Ag/AgCl). A Clark-type or World Precision Instrument O₂ electrode is attached to both the anode and cathode sections. Flow of fresh anaerobic media through the anodic compartment is accomplished using peristaltic pumps at a flow rate of 0.05 ml/min.

Similar experiments are performed with wild-type, single, double, triple quadruple, quintuple, and multiple mutant strains. Current and voltage output are monitored using a LabJack system as described above herein or an Agilent 34970-A data acquisition system that is linked to electronic databases. This system allows monitoring of current in the micro-ampere range and voltage in the micro to millivolt range.

### Example 9

### P. aeruginosa Mutant Strains

The PAO1 strain was used to prepare mutant strains. pilT, bdlA, nirS, and lasI single disruption mutations were constructed. pilT bdlA, bdlA nirS, bdlA lasI, nirS pilT, nirS lasI, and lasI pilT double disruption mutant strains were constructed. pilT bdlA nirS, bdlA lasI pilT, nirS lasI bdlA triple disruption mutant strains were constructed. A pilT bdlA nirS lasI quadruple disruption mutant strain was constructed.

A PAO1 strain stably comprising araBAD-ftsZ was constructed. The PAO1 araBAD-ftsZ strain was used to prepare mutant strains. pilT, bdlA, nirS, and lasI single disruption mutations were constructed in the PAO1 araBAD-ftsZ background. pilT bdlA, bdlA nirS, bdlA lasI, nirS pilT, nirS lasI, and lasI pilT double disruption mutations were constructed in the PAO1 araBAD-ftsZ background. pilT bdlA nirS, bdlA lasI pilT, and nirS lasI bdlA triple disruption mutations were constructed in the PAO1 araBAD-ftsZ background. A pilT bdlA nirS lasI quadruple disruption mutant strain was constructed in the PAO1 araBAD-ftsZ background.

A PAO1 strain stably comprising araBAD-ftsZ and a siRNA construct (PA0730) was constructed. The PAO1 araBAD-ftsZ PA0730 strain was used to prepare mutant strains. pilT, bdlA, nirS, and lasI single disruption mutations were constructed in the PAO1 araBAD-ftsZ PA0730 background. pilT bdlA, bdlA nirS, bdlA lasI, nirS pilT, nirS lasI, and lasI pilT double disruption mutations were constructed in the PAO1 araBAD-ftsZ PA0730 background. pilT bdlA nirS, bdlA lasI pilT, and nirS lasI bdlA triple disruption mutations were constructed in the PAO1 araBAD-ftsZ PA0730 background. A pilT bdlA nirS lasI quadruple disruption mutant strain was constructed in the PAO1 araBAD-ftsZ PA0730 background.

All publications, patents, and patent applications mentioned in the specification are indicative of the level of those skilled in the art to which this invention pertains. All publications, patents, and patent applications are herein incorporated by reference to the same extent as if each individual publication or patent application was specifically and individually incorporated by reference.

What is claimed is:

1. A system to prevent unauthorized use or release into the environment of genetically modified organisms or cells which comprises a) a genetically modified organism or cell and b) one or more keys, wherein contacting the genetically modified organisms or cell with the key results in viability or unviability of the genetically modified organism or cell.

2. The system of claim 1, wherein the genetically modified cell is a microorganism

3. The system of claim 2, wherein the microorganism is Psuedomonas aeruginosa.

4. The system of claim 1, wherein the key is a chemical inducer.

5. The system of claim 3, wherein the key is selected from the group consisting of arabinose and threonine.


SEQUENCE LISTING (38 sequences)

SEQ ID NO: 1 - DNA, 1035 bp, Pseudomonas aeruginosa
atggatattaccgagctgctcgccttcagtgccaaacagggcgcttcggacctgcacctctccgccggcctgccacccatgatccgggtggatggcgatgtacgccggatcaacctgccaccgctggaacacaagcaggtgcatgcgctgatctacgacatcatgaacgacaagcagcgcaaggacttcgaggaattcctcgagaccgacttctccttcgaggtgccgggcgtcgcgcgtttccgggtcaacgccttcaaccagaaccgtggcgccggcgcggtattccggaccattccctccaaggtactgaccatggaggagcttggcatgggagaagtgttcaaacgtgtttcagacgtcccgcgcgggttggtactggtcaccgggccgaccggttcgggcaagtccaccaccctggcggcgatgctcgattacctgaacaacaccaagtaccaccacatcctcaccatcgaggacccgatcgaattcgtccacgaatcgaagaagtgcctggtcaaccagcgcgaggtgcatcgcgacaccctcggcttcagcgaagcgctgcgctcggcgctgcgggaggacccggacatcatcctggtcggcgagatgcgcgacctggaaaccatccgcctggccctgaccgcggcggagaccggccacctggtattcggcaccctgcacaccacctcggcggcgaagaccatcgaccgggtggtcgacgtgttcccggccgaggaaaaggccatggttcgctcgatgctctccgagtcgctgcaatcggtgatctcgcagaccctgatcaagaagatcggcggcggccgggtggcggcccacgagatcatgatcggcaccccggcgatccgcaacctgatccgcgaggacaaggtcgcgcagatgtattcggcgatccagaccggcggctcgctgggcatgcagaccctcgacatgtgcctcaagggcctggtcgccaagggcctgatcagccgcgagaacgcccgcgagaaggcgaagatcccggaaaacttctga

SEQ ID NO: 2 - DNA, 450 bp, Pseudomonas aeruginosa
atgaaagctcaaaaaggctttaccttgatcgaactgatgatcgtggttgcgatcatcggtatcctggcggcaattgccattccccagtatcagaactatgttgcgcgttcggaaggtgcttcggcgctggcgacgatcaacccgctgaagaccactgttgaagagtcgctgtcgcgtggaattgctggtagcaaaattaaaattggtactactgcttctactgcgaccgaaacatatgtcggcgtcgagccggatgccaacaagttgggtgtaattgctgtagcaatcgaagatagtggtgcgggtgatattacctttaccttccagactggtacctctagtcccaagaatgctactaaagttatcactctgaaccgtactgcggatggggtctgggcttgtaaatctacccaggatccgatgttcactccgaaaggttgtgataactaa

SEQ ID NO: 3 - DNA, 1707 bp, Pseudomonas aeruginosa
atgccatttggcaagccactggtgggcaccttgctcgcctcgctgacgctgctgggcctggccaccgctcacgccaaggacgacatgaaagccgccgagcaataccagggtgccgcttccgccgtcgatcccgctcacgtggtgcgcaccaacggtgcccccgacatgagtgaaagcgagttcaacgaggccaagcagatctacttccaacgctgcgccggttgccacggcgtcctgcgcaagggcgccaccggcaagccgctgaccccggacatcacccagcaacgcggccagcaatacctggaagcgctgatcacctacggcaccccgctgggcatgccgaactggggcagctccggcgagctgagcaaggaacagatcaccctgatggccaagtacatccagcacaccccgccgcaaccgccggagtggggcatgccggagatgcgcgaatcgtggaaggtgctggtgaagccggaggaccggccgaagaaacagctcaacgacctcgacctgcccaacctgttctcggtgaccctgcgcgacgccgggcagatcgccctggtcgacggcgacagcaagaagatcgtcaaggtcatcgataccggctatgccgtgcatatctcgcggatgtccgcttccggccgctacctgctggtgatcggccgcgacgcgcggatcgacatgatcgacctgtgggccaaggagccgaccaaggtcgccgagatcaagatcggcatcgaggcgcgctcggtggaaagctccaagttcaagggctacgaggaccgctacaccatcgccggcgcctactggccgccgcagttcgcgatcatggacggcgagaccctggaaccgaagcagatcgtctccacccgcggcatgaccgtagacacccagacctaccacccggaaccgcgcgtggcggcgatcatcgcctcccacgagcaccccgagttcatcgtcaacgtgaaggagaccggcaaggtcctgctggtcaactacaaggatatcgacaacctcaccgtcaccagcatcggtgcggcgccgttcctccacgacggcggctgggacagcagccaccgctacttcatgaccgccgccaacaactccaacaaggttgccgtgatcgactccaaggaccgtcgcctgtcggccctggtcgacgtcggcaagaccccgcacccggggcgtggcgccaacttcgtgcatcccaagtacggcccggtgtggagcaccagccacctgggcgacggcagcatctcgctgatcggcaccgatccgaagaaccatccgcagtacgcctggaagaaagtcgccgaactacagggccagggcggcggctcgctgttcatcaagacccatccgaagtcctcgcacctctacgtcgacaccaccttcaaccccgacgccaggatcagccagagcgtcgcggtgttcgacctgaagaacctcgacgccaagtaccaggtgctgccgatcgccgaatgggccgatctcggcgaaggcgccaagcgggtggtgcagcccgagtacaacaagcgcggcgatgaagtctggttctcggtgtggaacggcaagaacgacagctccgcgctggtggtggtggacgacaagaccctgaagctcaaggccgtggtcaaggacccgcggctgatcaccccgaccggtaagttcaacgtctacaacacccagcacgacgtgtactga

SEQ ID NO: 4 - DNA, 1254 bp, Pseudomonas aeruginosa
atggcggccctggaccgctccatggcgcgcgtggagttcgatccggacggcaatatcaccgatgccaatgagaatttcctgaccctgctgggctaccgccgcgacgagatcctcggcaaaccgcatcgccagctttgcgacggcgcctacgcgcaatcggaagactaccggcgcttctgggaacgcctgcggcgcggcgaacacttttccggccgctgcaagcgcattacccgcgagggccggccactctggctggaagccacctacaaccccgtacgcgacgggcagggtcgactgctcaaggtggtcaagtacgccagcgacatcgatgccatcgtccaccaggaacacgagatgcagagcaagctggatgccctgtcccgctcgatggcgatgatcgagttcgacctcgacggcaatgtcctcgcggccaacgacaacttcctcgccaccatgggctatggccgggccgagctggccagcgccaaccaccgtcagttctgcgaaccgggctaccgcgacggcccacagtacgccgacctctggcgccgcctgaaccgcggcgagtacgtcaccgggcagttccgccgggtccaccgcaacggccagccggtctggctggaagccagctacaacccggtctacgacgccgacggcaagctctacaaggtggtcaagttcgccagcgatgtcagcgaccgcatgcgccgctaccaggccgaggcggacaacgcccaccaggcccataccctgtccaccgagacccgcacggtcgccgaacacggcgcgctgatcatccagagcgcggtggaggaaatgctcaagatcgcgaataccctggatgcttcctcgctgaacatcggcgaactgtcacagcactcgcaacagatcacctcgatcgtcaacaccatccgcgagatcgccgagcagaccaacctgctcgccctcaatgccgccatcgaggccgcccgcgccggcgaccagggtcgcggcttcgccgtggtggccgacgaggtgcggcaactggcggaacgcaccagcaagtcgaccaaggagatcgccgacatgatcggtcgcatccagaccggcacccgcagcgtcatcgacgacatgcagcacagccaggaacaggcgcggcgcggcgtggagctggccaacgaggccggcgcggcgatccttggcatccgcgagagcacgcacaaggtggtagaagcggtgcagcagttctcgcgcaccctcaacgccgatctctag

SEQ ID NO: 5 - DNA, 606 bp, Pseudomonas aeruginosa
atgatcgtacaaattggtcggcgcgaagagttcgataaaaaactgctgggcgagatgcacaagttgcgtgctcaagtgttcaaggagcgcaaaggctgggacgttagtgtcatcgacgagatggaaatcgatggttatgacgcactcagtccttattacatgttgatccaggaagatactcctgaagcccaggttttcggttgctggcgaattctcgataccactggcccctacatgctgaagaacaccttcccggagcttctgcacggcaaggaagcgccttgctcgccgcacatctgggaactcagccgtttcgccatcaactctggacagaaaggctcgctgggcttttccgactgtacgctggaggcgatgcgcgcgctggcccgctacagcctgcagaacgacatccagacgctggtgacggtaaccaccgtaggcgtggagaagatgatgatccgtgccggcctggacgtatcgcgcttcggtccgcacctgaagatcggcatcgagcgcgcggtggccttgcgcatcgaactcaatgccaagacccagatcgcgctttacgggggagtgctggtggaacagcgactggcggtttcatga

SEQ ID NO: 6 - DNA, 720 bp, Pseudomonas aeruginosa
atggccttggttgacggttttcttgagctggaacgctcaagtggaaaattggagtggagcgccatcctgcagaagatggcgagcgaccttggattctcgaagatcctgttcggcctgttgcctaaggacagccaggactacgagaacgccttcatcgtcggcaactacccggccgcctggcgcgagcattacgaccgggctggctacgcgcgggtcgacccgacggtcagtcactgtacccagagcgtactgccgattttctgggaaccgtccatctaccagacgcgaaagcagcacgagttcttcgaggaagcctcggccgccggcctggtgtatgggctgaccatgccgctgcatggtgctcgcggcgaactcggcgcgctgagcctcagcgtggaagcggaaaaccgggccgaggccaaccgtttcatggagtcggtcctgccgaccctgtggatgctcaaggactacgcactgcagagcggtgccggactggccttcgaacatccggtcagcaaaccggtggttctgaccagccgggagaaggaagtgttgcagtggtgcgccatcggcaagaccagttgggagatatcggttatctgcaactgctcggaagccaatgtgaacttccatatgggaaatattcggcggaagttcggtgtgacctcccgccgcgtagcggccattatggccgttaatttgggtcttattactctctga

SEQ ID NO: 7 - DNA, 1185 bp, Pseudomonas aeruginosa
atgtttgaactggtcgataacatcgcacaaaccgccgtcataaaagtgatcggtgtaggtggtggtggcggcaacgccgtcaatcacatggccaagaacaacgtcgagggcgtcgagttcatctgcgccaacaccgacgcacaagcgctgaagaacatcgcggcgcgcaccgttctgcaactcggcccgggcgtcaccaaggggctgggtgccggcgccaatccggaagtcggtcgtcaggcggctctggaagatcgcgagcgcatttccgaagtgctggaaggcgccgacatggtcttcatcaccaccggcatgggtggcggcaccggtaccggcgccgcgccgatcatcgccgaagtggcgaaggaaatgggcatcctcaccgtcgcggtggtgacccgcccgttcccgttcgaaggtcgcaagcgcatgcagatcgccgacgagggcatccgcgcgctggccgagagcgtcgattcgctgatcaccatcccgaacgagaagctgctgaccatcctcggcaaggacgccagcctgctggccgccttcgccaaggccgatgacgtgctggccggtgccgtgcgcggtatctccgacatcatcaagcgtccgggcatgatcaacgtcgacttcgccgacgtgaagaccgtcatgagcgaaatgggcatggcgatgatgggtaccggctgcgccagcggtccgaaccgtgcccgcgaggccaccgaggcggcaatccgcaacccgctgctggaagacgtcaacctgcagggcgcgcgcggcatcctggtgaacatcaccgcgggtccggacctgtccctgggcgagtactccgatgtcggcaacatcatcgaacagttcgcttccgagcacgccactgtgaaggtgggcaccgtgatcgacgcggacatgcgcgatgagctgcacgtcaccgtagtcgccaccggcctgggcgcgcgcctggagaaaccggtgaaggtcgtcgacaacaccgtgcagggcagtgcagcccaggcagccgctccggcccagcgcgagcagcagtcggtgaactaccgcgacctcgaccgtcctaccgtgatgcgcaaccagtctcacggcagcgcggcgaccgcggccaagctgaacccgcaggatgacctggattacctggatatcccggcgttcctgcgtcgtcaggccgattga

SEQ ID NO: 8 - DNA, 1467 bp, Pseudomonas aeruginosa
atggcccttacagtcaacacgaacattgcttccctgaacactcagcgcaacctgaatgcttcttccaacgacctcaacacctcgttgcagcgtctgaccaccggctaccgcatcaacagtgccaaggacgatgctgccggcctgcagatctccaaccgcctgtccaaccagatcagcggtctgaacgttgccacccgcaacgccaacgacggcatctccctggcgcagaccgctgaaggtgccctgcagcagtccaccaatatcctgcagcgtatccgcgacctggccctgcaatccgccaacggctccaacagcgacgccgaccgtgccgccctgcagaaagaagtcgctgcgcaacaggccgaactgacccgtatctccgataccaccaccttcggtggccgcaagctgctcgacggctccttcggcaccaccagcttccaggtcggttccaacgcctacgagaccattgacatcagcctgcagaatgcctctgccagcgccatcggttcttaccaggtcggcagcaacggcgcgggtaccgtcgccagcgtagcgggcaccgcgaccgcttcgggcatcgcctcgggcaccgtcaacctggtcggtggcggtcaggtgaagaacatcgccatcgccgccggcgatagcgccaaggccatcgccgagaagatggacggtgcgatcccgaacctgtcggctcgtgcccgtaccgtgttcaccgctgatgtcagcggcgtgaccggtggttcgctgaacttcgacgtaaccgttggcagcaacaccgtgagcctggcaggcgtgacctccactcaggatctggccgaccaactgaactccaactcgtcgaagctgggcatcactgccagcatcaacgacaagggtgtactgaccatcacctccgctaccggcgagaacgtcaagttcggtgcgcagaccggtaccgctactgccggtcaggtcgcagtgaaggtccagggttccgacggcaagttcgaagcggccgccaagaacgtggtagctgccggtactgccgctaccaccaccatcgtgaccggctacgtgcaactgaactcgccgaccgcctactcggtcagcggtaccggcacccaggcttcgcaggtcttcggcaacgccagcgccgcgcagaagagcagcgttgccagcgtcgacatctccactgccgacggcgcccagaacgccatcgcggtagtcgataacgccctggctgcgatcgacgcccagcgtgctgacctcggtgctgttcagaaccgcttcaagaacactatcgacaacctgaccaacatctcggaaaacgctaccaacgctcgtagccgcatcaaggacaccgacttcgctgccgaaaccgcggcgctgtcgaagaaccaggtgctgcaacaggccggtaccgcgatcctggcccaggccaaccagctgccgcaggcggtcctgagcctgctgcgctaa

SEQ ID NO: 9 - DNA, 2829 bp, Pseudomonas aeruginosa
gtggtacggcttcggatcgccataggactactcgtcagcttcctgctgcttctgctcggtccgatgtcgccggcggtcgcggacgacgccggcgtttccagcgttcccctgcaaaccaccgccactactcccagcgccaaccagaactggcgcctgctgcgcgacgagagcgcgcaactgcggatcgccgacgtcctccagcgcaaggagcagttccgcccgctggccaagcgctccttcatcttccccgccagcccccaggcggtctggctgcaggtccagcttcccgcgcagaaggtgccgagctggctgtggatcttcgccccacgggtgcagtacctggactactacctggtgcaggacggccaactggtgcgggaccagcacaccggcgagtcgcgaccgttccaggagcgcccgctgccctcgcgctcctacctgttctcgctgccggtcgacggcaagccgatgaccctgtacgtgcggatgacctccaaccatccgctgatggcctggttcgaccagatcgacgaagccggcctggtcggcctggagaagcccgcctacgccttcggcatgctgctcggcggcatgctcctgctgctgatgtacaacctgatccgcttcgcctactcgcgcagcgccagcagcctgtggctggcggcggtgcacgcggcgctggcggtctgcgccgcggcgaacctggggctggtggcgttctggctgccgggactcaagttcaaccagtcgctgaccgccgacctcggcgccctcggcgccgcggtgagcctgctgtggttcgcctgcagcttcttccgcggcactgcggaaagccgcctgaaccgcctcctgcaaggcgaggcgctgttgatcctggcggtcggcgcgatcatcgccttcacccagcaactctggttcagttggctgatctacctgctggtgatcctcagttcgctcagcgtcccgctgatcgcggcctggcactggtatcgcggctaccagccggcgcgcctgatcgttgccgggatgatcgtgttcaacgccggcttcatggtgttcctgccggtactgttcggcaccaagcaactcgatccgggctggctggtgctcggcgtgttcagcttcgccaccctcgccgggctggtcctgagcgtctcgctcaccgaacgccagcggctgatccagcagctcaacctgcaacagcgcaccagcgaggcggcgcacaccgcggaattgcagaccaaggccgagttcctggccaagatcagccacgagatccgcacgcccatgaacggcgtgctgggcatgaccgaactgctcctcggcaccccgttgtcggcgaagcagcgcgactacgtgcagaccatccacagcgccggcaacgagctgctcacgctgatcaacgagatactcgacatctccaagctggagtccgggcagatcgagctggacgaagtgcagttcgacctcaacgcgttgatcgaggactgcctggatatcttccgggtcaaggccgagcagcagcgcatcgagctgatcagcttcacccagccgcaggtgccgcgggtcatcggcggcgatccgacgcgcctgcgccaggtggtcctgagcctgctggacaacgcgttcaagcagaccgaggaaggcgagatcctgctggtggtggccctcgacgaccagggcgagacgccacgcctgcgcatcgccgtgcaggacagcggccacccgttcgacgccaaggagcgcgaggcgctgctcaccgccgagctgcacagcggcgacttcctttccgccagcaagctcggcagccacctcggcctgatcatcgcccgccaactggtgcggctgatgggcggggagttcggcatccagtccggcagcagccagggcaccaccctgtcgctgaccctgccgctcgacccgcagcagctggagaaccccaccgccgacctcgacggcccgctgcagggcgcccggctgctggtggtggacgacaacgaaacctgccgcaaggtgctggtgcagcagtgcagcggctggggcctgaacgtcagcgcggtgtcgtccggcaaggaggcgctggcccagttgcgcaccaaggcgcacctgcgcgagtacttcgacgtggtcctgctcgaccaggacatgcccggcatgaccggcatgcaactggcggcgaagatcaaggaagaccccaacctcaaccacgacatcctgctgatcatgctcaccgggatcagcaacgcgccgagcaagatcatcgcccgcaacgccgggatcaagcgcatcctggccaagccggtggccggctacacgctgaaggcgaccctcgccgacgaactcgcccagcgcggcgtcagcggcgtcaccaactacctgcaaccggccaaggaagcgcaggcgccgagcctgccgagcgacttccggatcctcgtcgccgaggacaacagcatctccaccaaggtcatccgcggcatgttgaacaagctcaacctccagccggacaccgccagcaacggccaggaggcgctcagcgcgatgaaggcgacccagtacgacctggtgctgatggactgcgagatgccggttctggacggcttctccgccaccgagcagctccgcgcctgggaagcccacgaacagcgcccgcataccccggtggtggcgctcaccgcgcacatcctcagcgaacacaaggaacgcgcgcggctggtcggcatggacgggcacatggccaagccggtggagctgtcgcaattgcgtgagctgatcgcctactgggtcggcgaacgcgatcgccgccgccagggcgacgccctgccctcctga

SEQ ID NO: 10 - DNA, 1113 bp, Pseudomonas aeruginosa
atgaagcgtgtaggtctgatcggttggcgcgggatggtgggttccgtactcatgcagcgaatgctggaagagcgggacttcgacctgatcgagccggtgttcttcaccacgtcgaacgtcggtggccagggcccggaagtgggcaaggacatcgctccgctgaaggacgcctacagcatcgacgagctgaagaccctcgacgtgatcctgacctgccagggcggcgactacaccagcgaagtattccccaagctgcgcgaggccggctggcagggctactggatcgatgccgcctccagtctgcgcatggaagacgacgcggtgatcgtcctcgacccggtcaaccgcaaggtcatcgaccaggctctggacgccggcacccgcaactacatcggcggcaactgcaccgtcagcctgatgctgatggccctgggcgggctgttcgacgccggtctggtcgagtggatgagtgccatgacctaccaggccgcctccggcgccggcgcgcagaacatgcgtgaactgctcaagcagatgggcgccgcccatgcatcggtggcggatgacctggccaacccggccagcgcgatcctcgacatcgaccgcaaggtggcggagaccctgcgcagcgaagccttccccaccgagcatttcggcgcgccgctgggcggcagcctgatcccctggatcgacaaggaactgcccaacgggcagagccgcgaagagtggaaggcccaggcggagaccaacaagatccttgcccgcttcaagaacccgatcccggtcgacggtatctgcgtgcgcgtcggcgccatgcgttgccacagccaggcgctgaccatcaagttgaacaaggatgtgccgctgaccgacatcgagggcctgatcagccagcacaatccctgggtcaagctggtgccgaaccaccgcgaggtaagcgtgcgcgagctgaccccggccgccgtcaccggcaccctgagcgttccggtcgggcgcctgcgcaagttgaacatgggctcccagtatctcggcgccttcaccgtcggcgaccagttgctgtggggcgctgccgagccgctgcggcgcatgttgcgcatcctgctggagcgctga

SEQ ID NO: 11 - DNA, 320 bp, Artificial sequence
  promoter
tccgcggcgcaagcaccgctgggcgtgaggggcaatgccttttgtaagcgcatgttgcttttgtccctatgtttcatccaactattgccagtctcttcgaaggttgccgaagtgtaaccgctaattgtccgtggagaatattcattaccgacttgtaattcttccgtcaccttgccgataagccccgctacctaacatctgccgcatcccggaagtgcaacttcgaatatcgccggcgagatatcgccgatgaacggttgcgtggttccgggccggtaatggatcgccgtcgccgacgacgatcagcagacgaggagcgg

SEQ ID NO: 12 - DNA, 23 bp, Artificial sequence
  primer
cgggatccgcggcgcaagcaccg

SEQ ID NO: 13 - DNA, 28 bp, Artificial sequence
  primer
cgtataccatatgccgctcctcgtctgc

SEQ ID NO: 14 - DNA, 28 bp, Artificial sequence
  primer
cgtataccatatgaagcgtgtaggtctg

SEQ ID NO: 15 - DNA, 26 bp, Artificial sequence
  primer
cggaattctcagcgctccagcaggat

SEQ ID NO: 16 - DNA, 17 bp, Artificial sequence
  primer
gtcgccgccggcgatgc

SEQ ID NO: 17 - DNA, 20 bp, Artificial sequence
  primer
cctcgttcccagtttgttcc

SEQ ID NO: 18 - DNA, 17 bp, Artificial sequence
  primer
cgccctatagtgagtcg

SEQ ID NO: 19 - DNA, 17 bp, Artificial sequence
  primer
cgccccaacctcgctgg

SEQ ID NO: 20 - DNA, 959 bp, Pseudomonas aeruginosa
aggccgatgcggtgctgctgggcgcggtaggggggccgaaatgggacaggatcgaacgcgatatccgtcccgagcgcggtctgctgaagattcgctcgcaactgggcctgttcgccaacctgcgtccggccatcctctatccgcaactggccgcggcctccagcctgaagccggaagtggtcgccgggctggacatcctcatcgtccgcgaactgaccggcggcatctacttcggccagccccgcgagcagcgcgtgctggagaacggcgagcgccaggcctacgacaccctgccgtacagcgagagcgaaatccgtcgtatcgcccgcgtcggtttcgacatggcccgcgtacgcaacaacaggctctgctcggtggacaaggccaacgttctcgcctccagccaactgtggcgggaagtggtggaagaggtggcgaaggactacccggacgtcgagctttcccatatgtacgtggataacgccgccatgcaactggtgcgcgcgcccaagcagttcgacgtgatggtcaccgacaacatgttcggcgacatcctgtcggatgaggcttccatgctgaccggttccatcggcatgctgccttctgcctcgctcgacgcgaacaacaagggcatgtacgaaccctgccacggttccgccccggacatcgccggtcagggcatcgccaacccgttggcgaccatcctctcggtctcgatgatgttgcgctacagcttcagccaggcgaccgccgccgacgccatcgagcaggcggtgagcaaggtcctcgatcaggggctgcgcaccggcgacatctggtccgaaggctgccgcaaggtcggcacccgggaaatgggcgatgccgtagtcgcggcactcaagaatctgtaatctgacgggcttggccggcgccatgatggctgccggccagctcacttctctcatataggtgtagttgcg

SEQ ID NO: 21 - DNA, 848 bp, Pseudomonas aeruginosa
tcgatccggcgccgtccgcaatgggcggcgcctcgattgcctgggcgaaggtgggcaagtaaagtgccacgcctatttcgcagaccgccgaggcttttcccatgtcccagcccctcaatgtcgcagtcgtaggcgccaccggatcggttggcgaggctctggtcggtctcctcgacgaacgcgacttcccgcttcatcgtttgcacctgctggccagcgccgagtcggccgggcagcgcatgggtttcgccgaaagcagcctgcgcgtcggcgatgtcgatagcttcgatttttccagtgtcggcctggccttcttcgccgcggcagccgaggtgtcgcgtgcccatgccgagcgcgcccgcgcggcgggttgttcggtgatcgacctgagcggcgccctggaaccgagcgtcgcaccgccggtcatggtttcggtgaatgccgaacgcctggcctcgcaagcggcgccgttccttttgtccagtccttgcgccgtagccgccgaactgtgcgaagtgctggcgccactcctggccacgctggattgccggcaactgaacctgacagcctgcctctccgtctccagcctggggcgcgaaggggtcaaggaactcgcccggcagaccgccgaactgctcaatgcgcgccccctggagccgcgtctcttcgaccgccagatcgccttcaacctgctcgcgcaggtcggagcggtggatgccgagggacacagcgcgatcgagcggcgcatcttcgcggaggtgcaggccttgcttggcgagcgtatcggaccgttgaacgttacctgcatccaggcgccggtattcttcggcgatagcctcagcgtgacc

SEQ ID NO: 22 - DNA, 26 bp, Artificial sequence
  primer
cgggatccaggccgatgcggtgctgc

SEQ ID NO: 23 - DNA, 26 bp, Artificial sequence
  primer
aactgcagcgcaactacacctatatg

SEQ ID NO: 24 - DNA, 26 bp, Artificial sequence
  primer
aactgcagtcgatccggcgccgtccg

SEQ ID NO: 25 - DNA, 27 bp, Artificial sequence
  primer
atagaattcggtcacgctgaggctatc

SEQ ID NO: 26 - DNA, 1464 bp, Escherichia coli
ttgttcgagtggagtccgccgtgtcactttcgctttggcagcagtgtcttgcccgattgcaggatgagttaccagccacagaattcagtatgtggatacgcccattgcaggcggaactgagcgataacacgctggccctgtacgcgccaaaccgttttgtcctcgattgggtacgggacaagtaccttaataatatcaatggactgctaaccagtttctgcggagcggatgccccacagctgcgttttgaagtcggcaccaaaccggtgacgcaaacgccacaagcggcagtgacgagcaacgtcgcggcccctgcacaggtggcgcaaacgcagccgcaacgtgctgcgccttctacgcgctcaggttgggataacgtcccggccccggcagaaccgacctatcgttctaacgtaaacgtcaaacacacgtttgataacttcgttgaaggtaaatctaaccaactggcgcgcgcggcggctcgccaggtggcggataaccctggcggtgcctataacccgttgttcctttatggcggcacgggtctgggtaaaactcacctgctgcatgcggtgggtaacggcattatggcgcgcaagccgaatgccaaagtggtttatatgcactccgagcgctttgttcaggacatggttaaagccctgcaaaacaacgcgatcgaagagtttaaacgctactaccgttccgtagatgcactgctgatcgacgatattcagttttttgctaataaagaacgatctcaggaagagtttttccacaccttcaacgccctgctggaaggtaatcaacagatcattctcacctcggatcgctatccgaaagagatcaacggcgttgaggatcgtttgaaatcccgcttcggttggggactgactgtggcgatcgaaccgccagagctggaaacccgtgtggcgatcctgatgaaaaaggccgacgaaaacgacattcgtttgccgggcgaagtggcgttctttatcgccaagcgtctacgatctaacgtacgtgagctggaaggggcgctgaaccgcgtcattgccaatgccaactttaccggacgggcgatcaccatcgacttcgtgcgtgaggcgctgcgcgacttgctggcattgcaggaaaaactggtcaccatcgacaatattcagaagacggtggcggagtactacaagatcaaagtcgcggatctcctttccaagcgtcgatcccgctcggtggcgcgtccgcgccagatggcgatggcgctggcgaaagagctgactaaccacagtctgccggagattggcgatgcgtttggtggccgtgaccacacgacggtgcttcatgcctgccgtaagatcgagcagttgcgtgaagagagccacgatatcaaagaagatttttcaaatttaatcagaacattgtcatcgtaaacctatgaaatttaccgtagaacgtgagcatttattaaaa

SEQ ID NO: 27 - DNA, 1902 bp, Escherichia coli
ataagtgtggataccgtcttatggagcaaaacccgcagtcacagctgaaacttcttgtcacccgtggtaaggagcaaggctatctgacctatgccgaggtcaatgaccatctgccggaagatatcgtcgattcagatcagatcgaagacatcatccaaatgatcaacgacatgggcattcaggtgatggaagaagcaccggatgccgatgatctgatgctggctgaaaacaccgcggacgaagatgctgccgaagccgccgcgcaggtgctttccagcgtggaatctgaaatcgggcgcacgactgacccggtacgcatgtacatgcgtgaaatgggcaccgttgaactgttgacccgcgaaggcgaaattgacatcgctaagcgtattgaagacgggatcaaccaggttcaatgctccgttgctgaatatccggaagcgatcacctatctgctggaacagtacgatcgtgttgaagcagaagaagcgcgtctgtccgatctgatcaccggctttgttgacccgaacgcagaagaagatctggcacctaccgccactcacgtcggttctgagctttcccaggaagatctggacgatgacgaagatgaagacgaagaagatggcgatgacgacagcgccgatgatgacaacagcatcgacccggaactggctcgcgaaaaatttgcggaactacgcgctcagtacgttgtaacgcgtgacaccatcaaagcgaaaggtcgcagtcacgctaccgctcaggaagagatcctgaaactgtctgaagtattcaaacagttccgcctggtgccgaagcagtttgactacctggtcaacagcatgcgcgtcatgatggaccgcgttcgtacgcaagaacgtctgatcatgaagctctgcgttgagcagtgcaaaatgccgaagaaaaacttcattaccctgtttaccggcaacgaaaccagcgatacctggttcaacgcggcaattgcgatgaacaagccgtggtcggaaaaactgcacgatgtctctgaagaagtgcatcgcgccctgcaaaaactgcagcagattgaagaagaaaccggcctgaccatcgagcaggttaaagatatcaaccgtcgtatgtccatcggtgaagcgaaagcccgccgtgcgaagaaagagatggttgaagcgaacttacgtctggttatttctatcgctaagaaatacaccaaccgtggcttgcagttccttgacctgattcaggaaggcaacatcggtctgatgaaagcggttgataaattcgaataccgccgtggttacaagttctccacctacgcaacctggtggatccgtcaggcgatcacccgctctatcgcggatcaggcgcgcaccatccgtattccggtgcatatgattgagaccatcaacaagctcaaccgtatttctcgccagatgctgcaagagatgggccgtgaaccgacgccggaagaactggctgaacgtatgctgatgccggaagacaagatccgcaaagtgctgaagatcgccaaagagccaatctccatggaaacgccgatcggtgatgatgaagattcgcatctgggggatttcatcgaggataccaccctcgagctgccgctggattctgcgaccaccgaaagcctgcgtgcggcaacgcacgacgtgctggctggcctgaccgcgcgtgaagcaaaagttctgcgtatgcgtttcggtatcgatatgaacaccgactacacgctggaagaagtgggtaaacagttcgacgttacccgcgaacgtatccgtcagatcgaagcgaaggcgctgcgcaaactgcgtcacccgagccgttctgaagtgctgcgtagcttcctggacgattaatcggtaggccggatcaggcgttacgccgcacccggcacta

SEQ ID NO: 28 - DNA, 574 bp, Escherichia coli
atgtctcgtgttgctaaagcaccggtcgttgttcctgccggcgttgacgtaaaaatcaacggtcaggttattacgatcaaaggtaaaaacggcgagctgactcgtactctcaacgatgctgttgaagttaaacatgcagataataccctgaccttcggtccgcgtgatggttacgcagacggttgggcacaggctggtaccgcgcgtgccctgctgaactcaatggttatcggtgttaccgaaggcttcactaagaagctgcagctggttggtgtaggttaccgtgcagcggttaaaggcaatgtgattaacctgtctctgggtttctctcatcctgttgaccatcagctgcctgcgggtatcactgctgaatgtccgactcagactgaaatcgtgctgaaaggcgctgataagcaggtgatcggccaggttgcagcggatctgcgcgcctaccgtcgtcctgagccttataaaggcaagggtgttcgttacgccgacgaagtcgtgcgtaccaaagaggctaagaagaagtaaggtaacactatggataagaaatctgctcgtatccgtcgtg

SEQ ID NO: 29 - DNA, 1050 bp, Escherichia coli
agtaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagagttcgatccgatcctgctgcgccctgttgacgatctggaattgactgtccgctctgctaactgccttaaagcagaagctatccactatatcggtgatctggtacagcgtaccgaggttgagctccttaaaacgcctaaccttggtaaaaaatctcttactgagattaaagacgtgctggcttcccgtggactgtctctgggcatgcgcctggaaaactggccaccggcaagcatcgctgacgagtaaccggatcacaggttaaggttttactgagaaggataaggtc

SEQ ID NO: 30 - DNA, 471 bp, Escherichia coli
ggcttcatagggagaataacatgagcattattaaagaatttcgcgaatttgcgatgcgcgggaacgtggtggatttggcggtgggtgtcattatcggtgcggcattcgggaagattgtctcttcactggttgccgatatcatcatgcctcctctgggcttattaattggcgggatcgattttaaacagtttgctgtcacgctacgcgatgcgcagggggatatccctgctgttgtgatgcattacggtgtcttcattcaaaacgtctttgattttctgattgtggcctttgccatctttatggcgattaagctaatcaacaaactgaatcggaaaaaagaagaaccagcagccgcacctgcaccaactaaagaagaagtattactgacagaaattcgtgatttgctgaaagagcagaataaccgctcttaacaagcgcctgaaagcagaagaccagtggtaaaaaagtgat

SEQ ID NO: 31 - DNA, 1104 bp, Escherichia coli
tttcaggattatcccttagtatgttgaaaaaatttcgtggcatgttttccaatgacttgtccattgacctgggtactgcgaataccctcatttatgtaaaaggacaaggcatcgtattgaatgagccttccgtggtggccattcgtcaggatcgtgccggttcaccgaaaagcgtagctgcagtaggtcatgacgcgaagcagatgctgggccgtacgccgggcaatattgctgccattcgcccaatgaaagacggcgttatcgccgacttcttcgtgactgaaaaaatgctccagcacttcatcaaacaagtgcacagcaacagctttatgcgtccaagcccgcgcgttctggtttgtgtgccggttggcgcgacccaggttgaacgccgcgcaattcgtgaatccgcgcagggcgctggtgcccgtgaagtcttcctgattgaagaaccgatggctgccgcaattggtgctggcctgccggtttctgaagcgaccggttctatggtggttgatatcggtggtggtaccactgaagttgctgttatctccttgaacggtgtggtttactcctcttctgtgcgcattggtggtgaccgtttcgacgaagctatcatcaactatgtgcgtcgtaattacggttctctgatcggtgaagccaccgcagaacgtatcaagcacgaaatcggttcggcttatccgggcgatgaagtccgtgaaatcgaagttcgtggccgtaacctggcagaaggtgttccacgcggttttaccctgaactccaatgaaatcctcgaagcactgcaggaaccgctgaccggtattgtgagcgcggtaatggttgcactggaacagtgcccgccggaactggcttccgacatctccgagcgcggcatggtgctcaccggtggtggcgcactgctgcgtaaccttgaccgtttgttaatggaagaaaccggcattccagtcgttgttgctgaagacccgctgacctgtgtggcgcgcggtggcggcaaagcgctggaaatgatcgacatgcacggcggcgacctgttcagcgaagagtaatcggatgcaggcaggggaagtgtctgtttaccctgcctgg

SEQ ID NO: 32 - DNA, 2523 bp, Escherichia coli
aaggtaacgaggtaacaaccatgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtatttccgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctgacatggttaaagtttatgccccggcttccagtgccaatatg

SEQ ID NO: 33 - DNA, 1237 bp, Pseudomonas aeruginosa
aagcatggcaaaccctatttcctggtccgcgccgacggcagcgatggcaacttcattcgctccgaccagccggacaagctgattccacaatgggagatcttcagctggtgatgtgcatttccaacctggcgaggccgcgctgacatgtcggtgttcaccccactcgaacgctcgaccctggaagccttcctcgccccctacgacctgggccgcctgcgggatttccgtggcatcgccgaaggctccgagaacagcaacttcttcgtcagcctggaacacggcgagttcgtcctgaccctggtcgagcgcggcccggtgcaggacctgccgttcttcatcgaactgctcgacgtgctccacgaggacggcctgccggtgccctacgccctgcgcacccgcgacggcgaggccctgcgccgtctcgaaggcaagccggcgctgctgcaaccgcgcctggccggccgccacgagcggcagccgaacgcccaccactgccaggaagtgggcgatctgctcggccaccttcacgccgccacccgcgggcgcatcctcgaacgcccgagcgaccgtggcctgccctggatgctggaacaaggtgccaacctcgccccgcgactaccggaacaggcccgcgccctgctcgccccggccctggcggagatcgccgcgctggatgccgagcgtccagcgctgccgcgcgccaacctgcatgccgacctgttccgcgacaacgtgctgttcgacggcccgcacctggccggcctgatcgacttctacaacgcctgctccggctggatgctctacgacctggcgatcaccctgaacgactggtgctccaataccgacggcagcctggacccggcccgcgcccgcgccctgctcgccgcctacgccaaccgccggccgttcaccgcgctggaagccgagcactggccgagcatgctgcgggtcgcctgcgtgcgcttctggctgtcgcgactgatcgccgccgaggcgttcgccggacaggacgtgctgatccatgatccggcggagttcgagatacgcctggcgcaacggcagaacgtggagattcatttgccgtttgcgttgtaggcaggaaccacaggagcacggccgtcctgcggccgttcgcccttcaaaaaccatgcggttcggcgtctcggagtgaggccgagccgacatcctgtcgaggcctcgtcgttcagcccacccccagcccggccatcctggccgg

SEQ ID NO: 34 - DNA, 804 bp, Pseudomonas aeruginosa
caggaataagccggcgttgccttgccctcggcgaggggctagagtgccgcgtccgtcgtcaccccatcgttcagagaatttcgaggagagtcctgtggaaattgcctgtcttgacctggaaggcgtgctggttccggaaatctggatcgcgttcgcggaaaaaaccggtatcgacgccttgaaggcgaccacccgggacattccggactacgacgtgctgatgaagcagcgcctgcgcatcctcgacgaacatggcctgaagctgggcgacatccaggaggtgatcgccaccctgaagccgctggagggggcggtggaattcgtcgactggctgcgcgagcgcttccaggtggtgattctctccgacaccttctacgagttctcccagccgctgatgcgccagctgggcttcccgaccctgttgtgccacaagctggagatcgacgacagcgaccgcgtggtcggctatcaattgcgccagaaggatcccaagcggcagtcggtgatcgccttcaagagcctctactatcgggtgatcgccgccggcgattcctacaacgacaccaccatgctttccgaggcccacgccggcatcctgttccatgccccggaaaacgtgatccgcgagtttccccagttccccgcggtgcatacctacgaagacctcaagcgcgagttcctcaaggcttccagccgttcgctgagcctgtagccgcgccgccgatgaaagagcccgcggaacgcgggctcttttctattccggggacgctgtcccggccattccttccagggtttccagcagca

SEQ ID NO: 35 - DNA, 843 bp, Escherichia coli
gttgtcatatggagtacaggatgtttgatatcggcgttaatttgaccagttcgcaatttgcgaaagaccgtgatgatgttgtagcgtgcgcttttgacgcgggagttaatgggctactcatcaccggcactaacctgcgtgaaagccagcaggcgcaaaagctggcgcgtcagtattcgtcctgttggtcaacggcgggcgtacatcctcacgacagcagccagtggcaagctgcgactgaagaagcgattattgagctggccgcgcagccagaagtggtggcgattggtgaatgtggtctcgactttaaccgcaacttttcgacgccggaagagcaggaacgcgcttttgttgcccagctacgcattgccgcagatttaaacatgccggtatttatgcactgtcgcgatgcccacgagcggtttatgacattgctggagccgtggctggataaactgcctggtgcggttcttcattgctttaccggcacacgcgaagagatgcaggcgtgcgtggcgcatggaatttatatcggcattaccggttgggtttgcgatgaacgacgcggactggagctgcgggaacttttgccgttgattccggcggaaaaattactgatcgaaactgatgcgccgtatctgctccctcgcgatctcacgccaaagccatcatcccggcgcaacgagccagcccatctgccccatattttgcaacgtattgcgcactggcgtggagaagatgccgcatggctggctgccaccacggatgctaatgtcaaaacactgtttgggattgcgttttagagtttgcggaactcggtattcttcacactgtgcttaatct

SEQ ID NO: 36 - DNA, 732 bp, Escherichia coli
atcaccgctaaggagtgattatggatctcgcgtcattacgcgctcaacaaatcgaactggcttcttctgtgatccgcgaggatcgactcgataaagatccaccggatctgatcgccggagccgatgtcgggtttgagcagggcggagaagtgacgcgagcggcgatggtgctgctgaaatatccctcgcttgagctggtcgagtataaagttgcccgcatcgccaccaccatgccttacattccaggttttctttccttccgcgaatatcctgcgctgctggcagcgtgggagatgctgtcgcaaaagccggatttagtgtttgtcgatggtcatgggatctcgcatcctcgccgtcttggcgtcgccagccattttggcttattggtggatgtgccgaccattggcgtggcgaaaaaacggctctgcggtaaattcgaaccgctctccagcgaaccgggcgcgctggccccactgatggataaaggcgagcagctggcctgggtctggcgcagcaaagcgcgctgtaacccgttgtttatcgctaccggccatcgggtcagcgtggacagcgcgctggcgtgggtacaacgctgcatgaaaggctatcgtctgccggagccaacgcgctgggcggacgcggtggcctcggaacgtccggcgttcgtgcgctatacagcaaatcagccctaattcaggtaaactgcggataatttccgtatttgagaactca

SEQ ID NO: 37 - DNA, 867 bp, Escherichia coli
gtatgagttccacacccattatgaaagcattctggcgtaacgccgcgttgctcgcggtttctctgcttcccttctcttctgccaacgccttagcgttgcaggcaaaacagtatggcgattttgatcgctatgtcctggccctctcctggcaaaccggattttgccagagtcaacacgatcgaaatcgtaacgaacgagatgaatgtcgcctgcaaaccgaaacgaccaacaaagctgattttctgaccgtacatggtctgtggccaggattgcctaaatcggttgctgcccgtggtgttgatgaacgccgctggatgcgcttcggttgcgctactcgcccaatcccgaatctaccagaagcgcgcgccagccgaatgtgttcatcgccggaaaccggattatcactggaaacggccgctaaactaagtgaagtcatgccaggagctggcggacgttcctgcctggaacgctacgaatatgccaaacacggtgcctgctttggttttgatccggacgcatacttcggtacgatggtacgcctgaatcaagaaattaaagagagcgaagccggaaaattccttgcggataattacggtaaaacagtgagccgccgtgactttgacgccgcctttgccaaaagctggggaaaagagaacgtgaaagcagttaagctaacgtgccagggtaaccctgcgtatttgactgaaattcagatctcgatcaaagctgacgccatcaacgctccgctttctgcaaactcattcttgccacaacctcacccaggtaactgtggcaaaacctttgtgattgataaagcgggttattaatcatctgatttacaaaacacgctaattcgtcttccataaa

SEQ ID NO: 38 - DNA, 3246 bp, Escherichia coli
agaataatgagtaagttacgatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtggtgaagaaaccaaaccgaccgagcaaccagcaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggctgaggtaacggaaaaagcgcgtaccgccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgatcgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgccgctcctgcgcgtccgcaacctgttgagtaataattagctcaaagtaatcaagccctggtaactgccaggg